(19) United States
(12) Patent Application Publication
(10) Pub. No.: US 2025/0354008 A1
(43) Pub. Date: Nov. 20, 2025

(54) BIO-DERIVED GREEN PARTICLE COMPOSITIONS AND METHODS THEREOF

(71) Applicant: MOMENTIVE PERFORMANCE MATERIALS INC., Niskayuna, NY (US)

(72) Inventors: Polina TSUPKO, Bronx, NY (US); Amar PAWAR, Briarcliff Manor, NY (US)

(21) Appl. No.: 19/209,579

(22) Filed: May 15, 2025

Related U.S. Application Data
(60) Provisional application No. 63/648,536, filed on May 16, 2024.

(51) Int. Cl. C08L 67/04; A61K 8/02; A61Q 1/06; A61Q 1/10; A61Q 1/12; A61Q 5/02; A61Q 17/04; A61Q 19/00

(52) U.S. Cl. CPC C08L 67/04; A61K 8/025; A61Q 1/06; A61Q 1/10; A61Q 1/12; A61Q 5/02; A61Q 17/04; A61Q 19/00; A61K 2800/412; A61K 2800/654

(57) ABSTRACT

The present disclosure provides a composition comprising polyhydroxyalkanoate (PHA) particles, wherein the PHA particles comprise at least one PHA polymer, and methods of preparing the PHA particles. These compositions of PHA particles are prepared by mixing at least one polyhydroxyalkanoate (PHA) polymer and at least one additional polymer that is not a PHA polymer. The present disclosure also provides methods of using the compositions of PHA particles in personal care compositions as well as personal care formulations comprising the personal care compositions.

### FIELD

The present disclosure provides a composition comprising polyhydroxyalkanoate (PHA) particles, wherein the PHA particles comprise at least one PHA polymer, and methods of preparing the PHA particles. These compositions of PHA particles are prepared by mixing at least one polyhydroxyalkanoate (PHA) polymer and at least one additional polymer that is not a PHA polymer. The present disclosure also provides methods of using the compositions of PHA particles in personal care compositions as well as personal care formulations comprising the personal care compositions.

### BACKGROUND

The personal care industry thrives on being able to deliver personal care formulations that provide multiple performance benefits based on mixtures of several components, with each component providing performance characteristics that impart an important and/or desirable effect to the final formulation. The use of compositions comprising particles are an important component for delivering these beneficial characteristics to compositions used in beauty and cosmetics products, especially in products left on the skin, face, and/or hair. The main role of these particles is to provide an improved texture to formulations and to achieve desirable optical effects such as line-blurring. Line-blurring, or soft focus effect, is an optical aberration caused by the particles maximizing the diffused light transmission and the scattered reflection, which leads to a minimization of the appearance of imperfections in the skin, face, and/or hair.

Particles currently available in the market and commonly used in personal care formulations comprise polysaccharides, polyesters, acrylates, and polyethylenes. Commercially available particles comprising polysaccharides and polyesters oftentimes absorb moisture and oils that change the size and refractive index of the particles. This change leads to undesirable effects in the formulations. Acrylates and polyethylenes fall under the category of microplastics, which are expected to be phased out in favor of more environmentally friendly products.

Due to their biodegradable and biocompatible properties, compositions comprising polyhydroxyalkanoate (PHA) particles would appear to be a potential substitute for use in personal care formulations. However, currently available PHA particles do not possess the attributes required for successful performance and enhanced sensory feel when used in a beauty and/or cosmetic product. To be successful in a beauty and/or cosmetic product, particles must not only be biodegradable, but they must also be spherical, have a smooth surface that is non-porous, and have a diameter of 1-30 μm.

Currently available PHA particles do not possess a diameter between about 1 μm and about 30 μm; rather, commercially available PHA particles are nano-sized and tend to form large aggregates. For example, U.S. 2014/0026916 discloses adding PHA microbeads with an average diameter of less than 400 μm to cosmetics formulations. The microbeads are specially designed for exfoliation and lack spherical shape. Large aggregates of nano-sized particles result in a worsened sensory feel and underperform in cosmetic formulations.

Russian Patent No. 2765717 describes a cosmetic composition which includes an oil phase and PHA particles produced by spray drying an aqueous suspension of PHA. The PHA particles used in the resultant cosmetic composition were porous and therefore, would not mix well with other components typically used in personal care compositions.

Thus, there is a need to provide biodegradable and biocompatible particles that are spherical, have a smooth surface that is non-porous, have a mean particle diameter between 1 μm and 30 μm, and that do not change in size or refractive index when added to personal care formulations.

### SUMMARY

The present disclosure provides a composition comprising polyhydroxyalkanoate (PHA) particles, wherein the PHA particles comprise at least one PHA polymer and wherein the PHA particles have a sphericity of at least 0.95, a solidity of at least 90%, and a Dv50 particle size from about 1 μm to about 50 μm.

In some aspects, the PHA polymer is a compound of Formula (I):

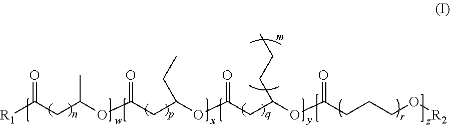

wherein:
w, x, y, and z are each independently an integer from 0 to 5000, wherein at least one of w, x, y, and z is ≥1;
m, n, p, q, and r are each independently an integer from 1 to 10; and
R₁ and R₂ are each independently H, OH, or CH₃.

In some aspects, the at least one PHA polymer has a Mw from about 500 g/mol to about 5,000,000 g/mol.

In some aspects, the at least one PHA polymer is selected from the group consisting of poly-3-hydroxybutyrate (PHB), poly-3-hydroxyvalerate (PHV), poly-3-hydroxyhexanoate (PHH), poly-3-hydroxyheptanoate, poly-3-hydroxyoctanoate, poly-3-hydroxynonanoate, poly-4-hydroxybutyrate (P4HB), poly(3-hydroxybutyrate-co-3-hydroxyvalerate) (PHBV), poly(3-hydroxybutyrate-co-3-hydroxyhexanoate) (PHBH), poly(3-hydroxybutyrate-co-4-hydroxybutyrate), poly(3-hydroxybuytrate-co-3-hydroxyvalerate-co-3-hydroxyhexanoate), poly-3-hydroxybutyrate, and poly(3-hydroxybutyrate-co-3-hydroxyvalerate).

In some aspects, the at least one PHA polymer is poly-3-hydroxybutyrate or poly(3-hydroxybutyrate-co-3-hydroxyvalerate).

In some aspects, the PHA particles have a Dv50 particle size from about 1 μm to about 20 μm.

In some aspects, the PHA particles have a solidity of at least 95%.

In some aspects, the PHA particles have a sphericity of at least 0.99.

In some aspects, the PHA particles have a particle size variation coefficient greater than 40.5%.

The present disclosure also provides a personal care composition comprising a composition of PHA particles.

In some aspects, the personal care composition further comprises at least one additional component selected from the group consisting of pigments, particulates, dyes, and combinations thereof.

In some aspects, the additional component is selected from the group consisting of iron oxides, zinc oxides, titanium dioxides, mica, silica, tapioca starch, rice starch, cellulose, cellulose acetate, polymethylsilsesquioxane, mica, and combinations thereof.

In some aspects, a personal care formulation comprises a composition of PHA particles or a personal care composition, wherein the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

The present disclosure also provides a use of the personal care composition in the manufacture of a personal care formulation.

The present disclosure also provides a method of preparing a composition of PHA particles comprising:
(a) mixing at least one PHA polymer and at least one additional polymer that is not a PHA polymer; and
(b1) melt-kneading the mixture of (a); or
(b2) heating the mixture of (a) for a time from about 1 minute to about 48 hours.

### BRIEF DESCRIPTION OF THE DRAWINGS

FIG. 1 shows a scanning electron microscopy (SEM) image of the poly(3-hydroxybutyrate) (PHB) particles prepared using the extrusion method of Example 1. The SEM image shows a PHB particle with a diameter of about 5 μm.

FIG. 2 shows a SEM image of the poly(3-hydroxybutyrate) (PHB) particles prepared using the extrusion method of Example 2. The SEM image shows PHB particles with a diameter of ≤10 μm.

FIG. 3 shows a spider chart comparing PHB particles, PHBV particles, and commercially available PHBV particles based on the following properties: spreadability, coarseness, glide, smoothness, and whitening.

FIG. 4 shows a SEM image of commercially available poly(3-hydroxybutyrate-co-3-hydroxyvalerate) (PHBV) particles. The SEM image shows these commercially available PHBV particles formed large aggregates with individual particles having a diameter of ≤1 μm.

### DETAILED DESCRIPTION

### I. Definitions

Unless otherwise indicated, any atom with unsatisfied valences is assumed to have hydrogen atoms sufficient to satisfy the valences.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the region of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

All ranges disclosed herein are inclusive of the endpoints, and the endpoints are independently combinable with each other. Each range disclosed herein constitutes a disclosure of any point or sub-range lying within the disclosed range. For example, the range from X to Y, is inclusive of X and Y. And, the range between X and Y, is inclusive of X and Y.

As used herein, the following definitions shall apply unless otherwise indicated. For purposes of the present disclosure, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March's Advanced Organic Chemistry,” 6th Ed., Smith, M. B. and March, J., eds. John Wiley & Sons, New York: 2007, the entire contents of which are hereby incorporated by reference in their entireties.

The phrase “personal care composition” as used herein, refers to a chemical mixture comprising a composition comprising PHA particles and additional components used for the purpose of cleansing, conditioning, grooming, beautifying, or otherwise enhancing the appearance of the human body.

The phrase “personal care formulation” as used herein, refers to a product marketed for use in its final form comprising a composition of PHA particles and/or a personal care composition, where the product has been further formulated for application to a human body. The personal care formulation can be in the form of a liquid, a gel, a foaming gel, a lotion, a cream, a paste, a powder, a bar, a stick, an emulsion, a dispersion, a suspension, a spray, or other commonly used forms in the industry.

The term “hydrocarbon,” as used herein by itself or as part of a group, refers to a straight- or branched-chain aliphatic series of one to two hundred carbon atoms, i.e., a C₁-C₂₀₀ hydrocarbon, or the number of carbon atoms designated, e.g., a C₁ hydrocarbon such as a methyl, a C₂ hydrocarbon such as ethyl, etc. In one embodiment, the hydrocarbon is a C₂-C₂₀₀ hydrocarbon group. In an embodiment, the hydrocarbon is a C₆-C₆₀ hydrocarbon group. In an embodiment, the hydrocarbon is a C₆-C₆₀ hydrocarbon group. In an embodiment, the hydrocarbon is a C₂-C₆₀ hydrocarbon group. In an embodiment, the hydrocarbon is a C₅-C₂₂ hydrocarbon group. Examples of hydrocarbon groups include butyl, octyl, decyl, lauryl, cetyl (palmityl), and stearyl.

The term “alkyl,” as used herein by itself or as part of a group, refers to a straight- or branched-chain aliphatic hydrocarbon containing one to two hundred carbon atoms, i.e., a C₂-C₂₀₀ alkyl, or the number of carbon atoms designated, e.g., a C₁ alkyl such as methyl, a C₂ alkyl such as ethyl, etc. In one embodiment, the alkyl is a C₂-C₂₀₀ alkyl group. In another embodiment, the alkyl is a C₆-C₆₀ alkyl group. In another embodiment, the alkyl is a C₂-C₆₀ alkyl group. In another embodiment, the alkyl is a C₅-C₂₂ alkyl group. Examples of alkyl groups include butyl, octyl, decyl, lauryl, cetyl (palmityl), and stearyl.

Various aspects of the disclosure are described in greater detail below.

### II. Compositions of Polyhydroxyalkanoate (PHA) Particles

The present disclosure provides improved compositions comprising PHA particles. The PHA particles possess a particle size sphericity and smoothness that allow these particles to blend well with other components commonly used in personal care compositions, to provide an enhanced sensory feel, and to display optical benefits observable by users such as line-blurring and soft focus effect.

In some aspects, the composition of PHA particles can comprise particles from at least one PHA polymer, wherein the PHA particles have a sphericity of at least 0.95, a solidity of at least 90%, and a Dv50 particle size from about 1 μm to about 50 μm.

In some aspects, the composition of PHA particles can comprise particles from at least one PHA polymer, wherein the PHA particles have a sphericity of at least 0.95.

In some aspects, the composition of PHA particles can comprise particles from at least one PHA polymer, wherein the PHA particles have a solidity of at least 90%.

In some aspects, the composition of PHA particles can comprise particles from at least one PHA polymer, wherein the PHA particles have a Dv50 particle size from about 1 μm to about 50 μm.

In some aspects, the composition of PHA particles comprises particles from at least one PHA polymer. In some aspects, the composition of PHA particles comprises particles from one PHA polymer. In some aspects, the composition of PHA particles comprises particles from two PHA polymers. In some aspects, the composition of PHA particles comprises particles from three PHA polymers. In some aspects, the composition of PHA particles comprises particles from four PHA polymers.

In some aspects, the at least one polyhydroxyalkanoate (PHA) polymer is a compound of Formula (I):

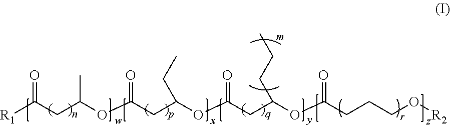

wherein:
w, x, y, and z are each independently an integer from 0 to 5,000, wherein at least one of w, x, y, and z is ≥1;
m, n, p, q, and r are each independently an integer from 1 to 10; and
R₁ and R₂ are each independently hydrogen, OH, or CH₃.

In some aspects, the at least one PHA polymer is a compound of Formula (I), wherein w, x, y, and z are each independently an integer from 1 to 5,000; m, n, p, q, and r are each independently an integer from 1 to 5; and R₁ and R₂ are each independently hydrogen, OH, or CH₃. In some aspects, the at least one PHA polymer is a compound of Formula (I), wherein w, x, y, and z are each independently an integer from 1 to 1,000; m, n, p, q, and r are each independently an integer from 1 to 5; and R₁ and R₂ are each independently hydrogen, OH, or CH₃. In some aspects, the at least one PHA polymer is a compound of Formula (I), wherein w, x, y, and z are each independently an integer from 1 to 500; m, n, p, q, and r are each independently an integer from 1 to 5; and R₁ and R₂ are each independently hydrogen, OH, or CH₃. In some aspects, the at least one PHA polymer is a compound of Formula (I), wherein w, x, y, and z are each independently an integer from 1 to 100; m, n, p, q, and r are each independently an integer from 1 to 5; and R₁ and R₂ are each independently hydrogen, OH, or CH₃.

In some aspects, the at least one PHA polymer is a compound of Formula (I), wherein w, x, y, and z are each independently an integer from 1 to 5,000; m, n, p, q, and r are each independently an integer from 1 to 3; and R₁ and R₂ are each independently hydrogen, OH, or CH₃. In some aspects, the at least one PHA polymer is a compound of Formula (I), wherein w, x, y, and z are each independently an integer from 1 to 1,000; m, n, p, q, and r are each independently an integer from 1 to 3; and R₁ and R₂ are each independently hydrogen, OH, or CH₃. In some aspects, the at least one PHA polymer is a compound of Formula (I), wherein w, x, y, and z are each independently an integer from 1 to 500; m, n, p, q, and r are each independently an integer from 1 to 3; and R₁ and R₂ are each independently hydrogen, OH, or CH₃. In some aspects, the at least one PHA polymer is a compound of Formula (I), wherein w, x, y, and z are each independently an integer from 1 to 100; m, n, p, q, and r are each independently an integer from 1 to 3; and R₁ and R₂ are each independently hydrogen, OH, or CH₃.

In some aspects, the at least one PHA polymer is a compound of Formula (I), wherein w, x, y, and z are each independently an integer from 1 to 5,000. In some aspects, the at least one PHA polymer is a compound of Formula (I), wherein w, x, y, and z are each independently an integer from 1 to 1,000. In some aspects, the at least one PHA polymer is a compound of Formula (I), wherein w, x, y, and z are each independently an integer from 1 to 500. In some aspects, the at least one PHA polymer is a compound of Formula (I), wherein w, x, y, and z are each independently an integer from 1 to 100.

In some aspects, the at least one PHA polymer is a compound of Formula (I), wherein m, n, p, q, and r are each independently an integer from 1 to 10. In some aspects, the at least one PHA polymer is a compound of Formula (I), wherein m, n, p, q, and r are each independently an integer from 1 to 7. In some aspects, the at least one PHA polymer is a compound of Formula (I), wherein m, n, p, q, and r are each independently an integer from 1 to 3. In some aspects, the at least one PHA polymer is a compound of Formula (I), wherein m, n, p, q, and r are each independently 1 or 2. In some aspects, the at least one PHA polymer is a compound of Formula (I), wherein m, n, p, q, and r are each independently 1.

In some aspects, the at least one PHA polymer is a compound of Formula (I), wherein w is an integer from 1 to 5,000; n is 1; R₁ is OH or CH₃; and R² is hydrogen. In some aspects, the at least one PHA polymer is poly-3-hydroxybutyrate (PHB).

In some aspects, the at least one PHA polymer is a compound of Formula (I), wherein x is an integer from 1 to 5,000; p is 1; R₁ is OH or CH₃; and R₂ is hydrogen. In some aspects, the at least one PHA polymer is poly-3-hydroxyvalerate (PHV).

In some aspects, the at least one PHA polymer is a compound of Formula (I), wherein y is an integer from 1 to 5,000; m and r are each independently 1; R₁ is OH or CH₃; and R₂ is hydrogen. In some aspects, the at least one PHA polymer is poly-3-hydroxyhexanoate (PHH).

In some aspects, the at least one PHA polymer is a compound of Formula (I), wherein z is an integer from 1 to 5,000; ris 1; R₁ is OH or CH₃; and R₂ is hydrogen. In some aspects, the at least one PHA polymer is poly-4-hydroxybutyrate (P4HB).

In some aspects, the at least one PHA polymer is a compound of Formula (I), wherein w and x are each independently an integer from 1 to 5,000; n and p are 1; R₁ is OH or CH₃; and R₂ is hydrogen. In some aspects, the at least one PHA polymer is poly(3-hydroxybutyrate-co-3-hydroxyvalerate) (PHBV).

In some aspects, the at least one PHA polymer is a compound of Formula (I), wherein w and y are each independently an integer from 1 to 5,000; m, n, and q are 1; R₁ is OH or CH₃; and R₂ is hydrogen. In some aspects, the at least one PHA polymer is poly(3-hydroxybutyrate-co-3-hydroxyhexanoate) (PHBH).

In some aspects, the at least one PHA polymer of Formula (I) is selected from the group consisting of poly-3-hydroxybutyrate (PHB), poly-3-hydroxyvalerate (PHV), poly-3-hydroxyhexanoate (PHH), poly-3-hydroxyheptanoate, poly-3-hydroxyoctanoate, poly-3-hydroxynonanoate, poly-4-hydroxybutyrate (P4HB), poly(3-hydroxybutyrate-co-3-hydroxyvalerate) (PHBV), poly(3-hydroxybutyrate-co-3-hydroxyhexanoate) (PHBH), poly(3-hydroxybutyrate-co-4-hydroxybutyrate), poly(3-hydroxybuytrate-co-3-hydroxyvalerate-co-3-hydroxyhexanoate), and combinations thereof. In some aspects, the PHA polymer of Formula (I) is selected from the group consisting of poly-3-hydroxybutyrate (PHB), and poly(3-hydroxybutyrate-co-3-hydroxyvalerate) (PHBV). In some aspects, the PHA polymer of Formula (I) is selected from the group consisting of poly-3-hydroxybutyrate (PHB), poly-3-hydroxyvalerate (PHV), poly-3-hydroxyhexanoate (PHH), poly-3-hydroxyheptanoate, poly-3-hydroxyoctanoate, poly-3-hydroxynonanoate, and poly-4-hydroxybutyrate (P4HB). In some aspects, the PHA polymer of Formula (I) is poly-3-hydroxybutyrate. In some aspects, the PHA polymer of Formula (I) is poly(3-hydroxybutyrate-co-3-hydroxyvalerate).

In some aspects, the at least one polyhydroxyalkanoate (PHA) polymer is a compound of Formula (II):

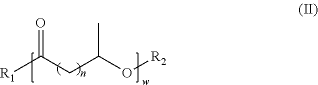

wherein:
w is an integer from 1 to 5,000;
n is an integer from 1 to 10; and
R₁ and R₂ are each independently hydrogen, OH, or CH₃.

In some aspects, the at least one PHA polymer is a compound of Formula (II), wherein w is an integer from 1 to 5,000; n is an integer from 1 to 5; and R₁ and R₂ are each independently hydrogen, OH, or CH₃. In some aspects, the at least one PHA polymer is a compound of Formula (II), wherein w is an integer from 1 to 1,000; n is an integer from 1 to 5; and R₁ and R₂ are each independently hydrogen, OH, or CH₃. In some aspects, the at least one PHA polymer is a compound of Formula (II), wherein w is an integer from 1 to 500; n is an integer from 1 to 5; and R₁ and R₂ are each independently hydrogen, OH, or CH₃. In some aspects, the at least one PHA polymer is a compound of Formula (II), wherein w is an integer from 1 to 100; n is an integer from 1 to 5; and R₁ and R₂ are each independently hydrogen, OH, or CH₃.

In some aspects, the at least one PHA polymer is a compound of Formula (II), wherein w is an integer from 1 to 5,000; n is an integer from 1 to 3; and R₁ and R₂ are each independently hydrogen, OH, or CH₃. In some aspects, the at least one PHA polymer is a compound of Formula (II), wherein w is an integer from 1 to 1,000; n is an integer from 1 to 3; and R₁ and R₂ are each independently hydrogen, OH, or CH₃. In some aspects, the at least one PHA polymer is a compound of Formula (II), wherein w is an integer from 1 to 500; n is an integer from 1 to 3; and R₁ and R₂ are each independently hydrogen, OH, or CH₃. In some aspects, the at least one PHA polymer is a compound of Formula (II), wherein w is an integer from 1 to 100; n is an integer from 1 to 3; and R₁ and R₂ are each independently hydrogen, OH, or CH₃.

In some aspects, the at least one PHA polymer is a compound of Formula (II), wherein w is an integer from 1 to 5,000. In some aspects, the at least one PHA polymer is a compound of Formula (II), wherein w is an integer from 1 to 1,000. In some aspects, the at least one PHA polymer is a compound of Formula (II), wherein w is an integer from 1 to 500. In some aspects, the at least one PHA polymer is a compound of Formula (II), wherein w is an integer from 1 to 100.

In some aspects, the at least one PHA polymer is a compound of Formula (II), wherein n is an integer from 1 to 10. In some aspects, the at least one PHA polymer is a compound of Formula (II), wherein n is an integer from 1 to 7. In some aspects, the at least one PHA polymer is a compound of Formula (II), wherein n is an integer from 1 to 3. In some aspects, the at least one PHA polymer is a compound of Formula (II), wherein n is 1 or 2. In some aspects, the at least one PHA polymer is a compound of Formula (II), wherein n is 1.

In some aspects, the at least one PHA polymer is a compound of Formula (II), wherein w is an integer from 1 to 5,000; n is 1; R₁ is OH or CH₃; and R₂ is hydrogen.

In some aspects, the at least one PHA polymer of Formula (II) is selected from the group consisting of poly-3-hydroxybutyrate (PHB), poly-3-hydroxyvalerate (PHV), poly-3-hydroxyhexanoate (PHH), poly-3-hydroxyheptanoate, poly-3-hydroxyoctanoate, and poly-3-hydroxynonanoate. In some aspects, the at least one PHA polymer of Formula (II) is selected from the group consisting of poly-3-hydroxybutyrate (PHB) and poly-3-hydroxyvalerate (PHV). In some aspects, the at least one PHA polymer of Formula (II) is poly-3-hydroxybutyrate (PHB).

In some aspects, the at least one polyhydroxyalkanoate (PHA) is a compound of Formula (III):

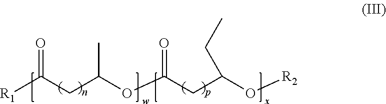

wherein:
w and x are each independently an integer from 1 to 5,000;
n and p are each independently an integer from 1 to 10; and
R₁ and R₂ are each independently hydrogen, OH, or CH₃.

In some aspects, the at least one PHA polymer is a compound of Formula (III), wherein w and x are each independently an integer from 1 to 5,000; n and p are each independently an integer from 1 to 5; and R₁ and R₂ are each independently hydrogen, OH, or CH₃. In some aspects, the at least one PHA polymer is a compound of Formula (III), wherein w and x are each independently an integer from 1 to 1,000; n and p are each independently an integer from 1 to 5; and R₁ and R₂ are each independently hydrogen, OH, or CH₃. In some aspects, the at least one PHA polymer is a compound of Formula (III), wherein w and x are each independently an integer from 1 to 500; n and p are each independently an integer from 1 to 5; and R₁ and R₂ are each independently hydrogen, OH, or CH₃. In some aspects, the at least one PHA polymer is a compound of Formula (III), wherein w and x are each independently an integer from 1 to 100; n and p are each independently an integer from 1 to 5; and R₁ and R₂ are each independently hydrogen, OH, or CH₃.

In some aspects, the at least one PHA polymer is a compound of Formula (III), wherein w and x are each independently an integer from 1 to 5,000; n and p are each independently an integer from 1 to 3; and R₁ and R₂ are each independently hydrogen, OH, or CH₃. In some aspects, the at least one PHA polymer is a compound of Formula (III), wherein w and x are each independently an integer from 1 to 1,000; n and p are each independently an integer from 1 to 3; and R₁ and R₂ are each independently hydrogen, OH, or CH₃. In some aspects, the at least one PHA polymer is a compound of Formula (III), wherein w and x are each independently an integer from 1 to 500; n and p are each independently an integer from 1 to 3; and R₁ and R₂ are each independently hydrogen, OH, or CH₃. In some aspects, the at least one PHA polymer is a compound of Formula (III), wherein w and x are each independently an integer from 1 to 100; n and p are each independently an integer from 1 to 3; and R₁ and R₂ are each independently hydrogen, OH, or CH₃.

In some aspects, the at least one PHA polymer is a compound of Formula (III), wherein w and x are each independently an integer from 1 to 5,000. In some aspects, the at least one PHA polymer is a compound of Formula (III), wherein w and x are each independently an integer from 1 to 1,000. In some aspects, the at least one PHA polymer is a compound of Formula (III), wherein w and x are each independently an integer from 1 to 500. In some aspects, the at least one PHA polymer is a compound of Formula (III), wherein w and x are each independently an integer from 1 to 100.

In some aspects, the at least one PHA polymer is a compound of Formula (III), wherein n and p are each independently an integer from 1 to 10. In some aspects, the at least one PHA polymer is a compound of Formula (III), wherein n and p are each independently an integer from 1 to 7. In some aspects, the at least one PHA polymer is a compound of Formula (III), wherein n and p are each independently an integer from 1 to 3. In some aspects, the at least one PHA polymer is a compound of Formula (III), wherein n and p are each independently 1 or 2. In some aspects, the at least one PHA polymer is a compound of Formula (III), wherein n and p are each independently 1.

In some aspects, the at least one PHA polymer is a compound of Formula (III), wherein w and x are each independently an integer from 1 to 5,000; n and p are 1; R₁ is OH or CH₃; and R₂ is hydrogen. In some aspects, the PHA is poly(3-hydroxybutyrate-co-3-hydroxyvalerate) (PHBV).

In some aspects, the at least one PHA polymer of Formula (III) is selected from the group consisting of poly(3-hydroxybutyrate-co-3-hydroxyvalerate) (PHBV), poly(3-hydroxybutyrate-co-3-hydroxyhexanoate) (PHBH), and poly(3-hydroxybutyrate-co-4-hydroxybutyrate).

In some aspects, the at least one polyhydroxyalkanoate (PHA) polymer is a compound of Formula (IV):

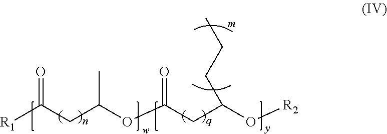

wherein:
w and y are each independently an integer from 1 to 5,000;
m, n, and q are each independently an integer from 1 to 10; and
R₁ and R₂ are each independently hydrogen, OH, or CH₃.

In some aspects, the at least one PHA polymer is a compound of Formula (IV), wherein w and y are each independently an integer from 1 to 5,000; m, n, and q are each independently an integer from 1 to 5; and R₁ and R₂ are each independently hydrogen, OH, or CH₃. In some aspects, the at least one PHA polymer is a compound of Formula (IV), wherein w and y are each independently an integer from 1 to 1,000; m, n, and q are each independently an integer from 1 to 5; and R₁ and R₂ are each independently hydrogen, OH, or CH₃.

In some aspects, the at least one PHA polymer is a compound of Formula (IV), wherein w and y are each independently an integer from 1 to 500; m, n, and q are each independently an integer from 1 to 5; and R₁ and R₂ are each independently hydrogen, OH, or CH₃. In some aspects, the at least one PHA polymer is a compound of Formula (IV), wherein w and y are each independently an integer from 1 to 100; m, n, and q are each independently an integer from 1 to 5; and R₁ and R₂ are each independently hydrogen, OH, or CH₃.

In some aspects, the at least one PHA polymer is a compound of Formula (IV), wherein w and y are each independently an integer from 1 to 5,000; m, n, and q are each independently an integer from 1 to 3; and R₁ and R₂ are each independently hydrogen, OH, or CH₃. In some aspects, the at least one PHA polymer is a compound of Formula (IV), wherein w and y are each independently an integer from 1 to 1,000; m, n, and q are each independently an integer from 1 to 3; and R₁ and R₂ are each independently hydrogen, OH, or CH₃. In some aspects, the at least one PHA polymer is a compound of Formula (IV), wherein w and y are each independently an integer from 1 to 500; m, n, and q are each independently an integer from 1 to 3; and R₁ and R₂ are each independently hydrogen, OH, or CH₃. In some aspects, the at least one PHA polymer is a compound of Formula (IV), wherein w and y are each independently an integer from 1 to 100; m, n, and q are each independently an integer from 1 to 3; and R₁ and R₂ are each independently hydrogen, OH, or CH₃.

In some aspects, the at least one PHA polymer is a compound of Formula (IV), wherein w and y are each independently an integer from 1 to 5,000. In some aspects, the at least one PHA polymer is a compound of Formula (IV), wherein w and y are each independently an integer from 1 to 1,000. In some aspects, the at least one PHA polymer is a compound of Formula (IV), wherein w and y are each independently an integer from 1 to 500. In some aspects, the at least one PHA polymer is a compound of Formula (IV), wherein w and y are each independently an integer from 1 to 100.

In some aspects, the at least one PHA polymer is a compound of Formula (IV), wherein m, n, and q are each independently an integer from 1 to 10. In some aspects, the at least one PHA polymer is a compound of Formula (IV), wherein m, n, and q are each independently an integer from 1 to 7. In some aspects, the at least one PHA polymer is a compound of Formula (IV), wherein m, n, and q are each independently an integer from 1 to 3. In some aspects, the at least one PHA polymer is a compound of Formula (IV), wherein m, n, and q are each independently 1 or 2. In some aspects, the at least one PHA polymer is a compound of Formula (IV), wherein m, n, and q are each independently 1.

In some aspects, the at least one PHA polymer is a compound of Formula (IV), wherein w and x are each independently an integer from 1 to 5,000; m is 2; n and q are 1; R₁ is OH or CH₃; and R₂ is hydrogen. In some aspects, the at least one PHA polymer of Formula (IV) is poly(3-hydroxybutyrate-co-3-hydroxyhexanoate) (PHBH).

In some aspects, the at least one polyhydroxyalkanoate (PHA) polymer is a compound of Formula (V):

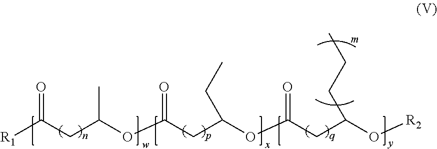

wherein:
w, x, and y are each independently an integer from 1 to 5,000;
m, n, p, and q are each independently an integer from 1 to 10; and
R₁ and R₂ are each independently hydrogen or CH₃.

In some aspects, the at least one PHA polymer is a compound of Formula (V), wherein w, x, and y are each independently an integer from 1 to 5,000; m, n, p, and q are each independently an integer from 1 to 5; and R₁ and R₂ are each independently hydrogen or CH₃. In some aspects, the at least one PHA polymer is a compound of Formula (V), wherein w, x, and y are each independently an integer from 1 to 1,000; m, n, p, and q are each independently an integer from 1 to 5; and R₁ and R₂ are each independently hydrogen or CH₃. In some aspects, the at least one PHA polymer is a compound of Formula (V), wherein w, x, and y are each independently an integer from 1 to 500; m, n, p, and q are each independently an integer from 1 to 5; and R₁ and R₂ are each independently hydrogen or CH₃. In some aspects, the at least one PHA polymer is a compound of Formula (V), wherein w, x, and y are each independently an integer from 1 to 100; m, n, p, and q are each independently an integer from 1 to 5; and R₁ and R₂ are each independently hydrogen or CH₃.

In some aspects, the at least one PHA polymer is a compound of Formula (V), wherein w, x, and y are each independently an integer from 1 to 5,000; m, n, p, and q are each independently an integer from 1 to 3; and R₁ and R₂ are each independently hydrogen or CH₃. In some aspects, the at least one PHA polymer is a compound of Formula (V), wherein w, x, and y are each independently an integer from 1 to 1,000; m, n, p, and q are each independently an integer from 1 to 3; and R₁ and R₂ are each independently hydrogen or CH₃. In some aspects, the at least one PHA polymer is a compound of Formula (V), wherein w, x, and are each independently an integer from 1 to 500; m, n, p, and q are each independently an integer from 1 to 3; and R₁ and R₂ are each independently hydrogen or CH₃. In some aspects, the at least one PHA polymer is a compound of Formula (V), wherein w, x, and y are each independently an integer from 1 to 100; m, n, p, and q are each independently an integer from 1 to 3; and R₁ and R₂ are each independently hydrogen or CH₃.

In some aspects, the at least one PHA polymer is a compound of Formula (V), wherein w, x, and y are each independently an integer from 1 to 5,000. In some aspects, the at least one PHA polymer is a compound of Formula (V), wherein w, x, and y are each independently an integer from 1 to 1,000. In some aspects, the at least one PHA polymer is a compound of Formula (V), wherein w, x, and y are each independently an integer from 1 to 500. In some aspects, the at least one PHA polymer is a compound of Formula (V), wherein w, x, and y are each independently an integer from 1 to 100.

In some aspects, the at least one PHA polymer is a compound of Formula (V), wherein m, n, p, and q are each independently an integer from 1 to 10. In some aspects, the at least one PHA polymer is a compound of Formula (V), wherein m, n, p, and q are each independently an integer from 1 to 7. In some aspects, the at least one PHA polymer is a compound of Formula (V), wherein m, n, p, and q are each independently an integer from 1 to 3. In some aspects, the at least one PHA polymer is a compound of Formula (V), wherein m, n, p, and q are each independently 1 or 2. In some aspects, the at least one PHA polymer is a compound of Formula (V), wherein m, n, p, and q are each independently 1.

In some aspects, the at least one PHA polymer is a compound of Formula (V), wherein w, x, and y are each independently an integer from 1 to 5,000; m, n, p, and q are 1; R₁ is CH₃; and R₂ is hydrogen.

In some aspects, the at least one PHA polymer of Formula (V) is poly(3-hydroxybuytrate-co-3-hydroxyvalerate-co-3-hydroxyhexanoate).

In some aspects, the at least one polyhydroxyalkanoate (PHA) polymer is a compound of Formula (VI):

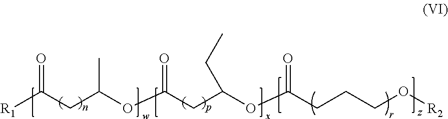

wherein:
w, x, and z are each independently an integer from 1 to 5,000;
n, p, and r are each independently an integer from 1 to 10; and
R₁ and R₂ are each independently hydrogen or CH₃.

In some aspects, the at least one PHA polymer is a compound of Formula (VI), wherein w, x, and z are each independently an integer from 1 to 5,000; n, p, and r are each independently an integer from 1 to 5; and R₁ and R₂ are each independently hydrogen or CH₃. In some aspects, the at least one PHA polymer is a compound of Formula (VI), wherein w, x, and z are each independently an integer from 1 to 1,000; n, p, and r are each independently an integer from 1 to 5; and R₁ and R₂ are each independently hydrogen or CH₃. In some aspects, the at least one PHA polymer is a compound of Formula (VI), wherein w, x, and z are each independently an integer from 1 to 500; n, p, and r are each independently an integer from 1 to 5; and R₁ and R₂ are each independently hydrogen or CH₃. In some aspects, the at least one PHA polymer is a compound of Formula (VI), wherein w, x, and z are each independently an integer from 1 to 100; n, p, and r are each independently an integer from 1 to 5; and R₁ and R₂ are each independently hydrogen or CH₃.

In some aspects, the at least one PHA polymer is a compound of Formula (VI), wherein w, x, and z are each independently an integer from 1 to 5,000; n, p, and r are each independently an integer from 1 to 3; and R₁ and R₂ are each independently hydrogen or CH₃. In some aspects, the at least one PHA polymer is a compound of Formula (VI), wherein w, x, and z are each independently an integer from 1 to 1,000; n, p, and r are each independently an integer from 1 to 3; and R₁ and R₂ are each independently hydrogen or CH₃. In some aspects, the at least one PHA polymer is a compound of Formula (VI), wherein w, x, and z are each independently an integer from 1 to 500; n, p, and r are each independently an integer from 1 to 3; and R₁ and R₂ are each independently hydrogen or CH₃. In some aspects, the at least one PHA polymer is a compound of Formula (VI), wherein w, x, and z are each independently an integer from 1 to 100; n, p, and r are each independently an integer from 1 to 3; and R₁ and R₂ are each independently hydrogen or CH₃.

In some aspects, the at least one PHA polymer is a compound of Formula (VI), wherein w, x, and z are each independently an integer from 1 to 5,000. In some aspects, the at least one PHA polymer is a compound of Formula (VI), wherein w, x, and z are each independently an integer from 1 to 1,000. In some aspects, the at least one PHA polymer is a compound of Formula (VI), wherein w, x, and z are each independently an integer from 1 to 500. In some aspects, the at least one PHA polymer is a compound of Formula (VI), wherein w, x, and z are each independently an integer from 1 to 100.

In some aspects, the at least one PHA polymer is a compound of Formula (VI), wherein n, p, and r are each independently an integer from 1 to 10. In some aspects, the at least one PHA polymer is a compound of Formula (VI), wherein n, p, and r are each independently an integer from 1 to 7. In some aspects, the at least one PHA polymer is a compound of Formula (VI), wherein n, p, and r are each independently an integer from 1 to 3. In some aspects, the at least one PHA polymer is a compound of Formula (VI), wherein n, p, and r are each independently 1 or 2. In some aspects, the at least one PHA polymer is a compound of Formula (VI), wherein n, p, and r are each independently 1.

In some aspects, the weight average molecular weight (M_{W}) of the at least one PHA polymer is from about 500 g/mol to about 2,000,000 g/mol. In some aspects, the weight average molecular weight of the at least one PHA polymer is from about 500 g/mol to about 2,000,000 g/mol, from about 10,000 g/mol to about 1,500,000 g/mol, from about 100,000 g/mol to about 1,250,000 g/mol, from about 500,000 g/mol to about 1,000,000 g/mol, from about 1,000,000 to about 2,000,000 g/ml. In some aspects, the weight average molecular weight of the at least one PHA polymer is about 500 g/mol, about 1,000 g/mol, about 2,500 g/mol, about 5,000 g/mol, about 10,000 g/mol, about 25,000 g/mol, about 50,000 g/mol, about 75,000 g/mol, about 100,000 g/mol, about 250,000 g/mol, about 500,000 g/mol, about 750,000 g/mol, about 1,000,000 g/mol, about 1,250,000 g/mol, about 1,500,000 g/mol, 1,750,000 g/mol, or about 2,000,000 g/mol. In some aspects, the weight average molecular weight of the at least one PHA polymer is about 240,000 g/mol. In some aspects, the weight average molecular weight of the at least one PHA polymer is about 280,000 g/mol. The weight average molecular weight is measured by gel permeation chromatography.

Sphericity is the measure of how spherical (round) an object is—it is a measure of the compactness measure of a shape. Wadell, H., “Volume, Shape, and Roundness of Quartz Particles,” J. of Geology 43:250-280 (1935), defines sphericity, Y′, of a particle as: the ratio of the surface area of the sphere (with the same volume as the given particle) to the surface area of the particle:

$$Ψ = ( {{π}^{1 / 3} ( 6 {V}_{p} )}^{2 / 3} ) / {A}_{p}$$

wherein Vₚ is the volume of the particle and Aₚ is the surface area of the particle. The sphericity of a sphere is 1 and any particle which is not a sphere will have sphericity of less than 1.

The sphericity of a PHA particle can be measured by the following method. Using a JCM-6000 benchtop Scanning Electron Microscope (SEM) manufactured by JEOL Ltd, Japan, an image of particles can be taken and the major axis and minor axis of 30 randomly selected particles can be measured. Then a minor axis/major axis ratio of each particle can be determined. The average value of the minor axis/major axis ratios for the 30 particles can be taken as the particle's sphericity. A particle is determined to be more spherical as the sphericity, or the average value of the minor axis/major axis ratio, gets closer to 1. A particle is considered to be spherical if its sphericity fall within the range of 0.98-1.

In some aspects, the polyhydroxyalkanoate (PHA) particles in the composition can have a sphericity from about 0.90 to about 1.0 as measured by SEM. In some aspects, the PHA particles in the composition can have a sphericity from about 0.95 to about 1.0. In some aspects, the PHA particles in the composition can have a sphericity from about 0.97 to about 1.0. In some aspects, the PHA particles in the composition can have a sphericity from about 0.98 to about 1. In some aspects, the PHA particles in the composition can have a sphericity of about 0.90, about 0.91, about 0.92, about 0.93, about 0.94, about 0.95, about 0.96, about 0.97, about 0.98, about 0.99, or about 1.0.

Solidity is the measure of how uniform or nonporous an object is—it is a measure of the solidity versus the porosity of a shape. Since solidity and porosity are complements of each other, the porosity (P) plus the solidity(S) should be 100%. Therefore S %=100%-P %. A value of S %=100% is an indication of a non-porous, completely solid, object. Solidity was measured using a JCM-6000 benchtop Scanning Electron Microscope (SEM) manufactured by JEOL Ltd, Japan. 30 random particles were assessed for the presence/absence of recessed areas using SEM. The recessed area is identified as any black colored area observed in SEM image of particles. Particles with no visible recessed areas were classified as solid and were given a solidity value(S) of 100%.

In some aspects, the polyhydroxyalkanoate (PHA) particles in the composition can have a solidity from about 90% to about 100% as measured by SEM. In some aspects, the PHA particles in the composition can have a solidity from about 92% to about 100%. In some aspects, the PHA particles in the composition can have a solidity from about 94% to about 100%. In some aspects, the PHA particles in the composition can have a solidity from about 96% to about 100%. In some aspects, the PHA particles in the composition can have a solidity from about 98% to about 100%. In some aspects, the PHA particles in the composition can have a solidity of about 90%, about 91%, about 92%, about 93%, about 94%, about 95%, about 96%, about 97%, about 98%, about 99%, or about 100%.

In some aspects, the polyhydroxyalkanoate (PHA) particles in the composition can have a porosity from 0% to about 10% as measured by SEM. In some aspects, the PHA particles in the composition can have a porosity from 0% to about 8%. In some aspects, the PHA particles in the composition can have a porosity from 0% to about 6%. In some aspects, the PHA particles in the composition can have a porosity from 0% to about 4%. In some aspects, the PHA particles in the composition can have a porosity from 0% to about 2%. In some aspects, the PHA particles in the composition can have a porosity of 0%, about 1%, about 2%, about 3%, about 4%, about 5%, about 6%, about 7%, about 8%, about 9%, or about 10%.

A particle size distribution can be expressed using four values. “D”, represents the percentage of the particles in the composition that are smaller than an indicated size. The analyzer used with laser diffraction to determine particle size does not measure the particles one by one, but rather uses light with different angles and then retrieves the diffraction patterns from image sensors. Then, by performing addition, subtraction, and cross-analysis calculations, the instrument determines the statistical proportion of the sizes of the particles.

The instrument also calculates the volume median diameter using software. The volume median diameter (Dv50) is the particle diameter for which 50% of the particle volume is contained in smaller particle diameter than Dv50 and 50% is contained in larger particle diameter than Dv50. Therefore, the Dv50 is half the total particle volume. Particle size distribution of PHA particles can be measured using the following method. PHA particles were suspended in cyclopentasiloxane and sonicated for 10 minutes. A cuvette was filled with cyclopentasiloxane and placed in the particle size analyzer, LA-960 laser diffraction particle size analyzer manufactured by Horiba Scientific, Japan. A few drops of the suspension containing PHA particles were then transferred into the cuvette with cyclopentasiloxane and stirred with a magnet. Once the transmittance reached the acceptable range, the measurement was performed.

A Dv10 value means 10% of the volume of particles is occupied by particles below diameter of Dv10. A Dv90 value means that 90% of the volume of particles in the composition are smaller than the Dv90. And, a Dv99 value means that 99% of the volume of particles in the composition are smaller than the Dv99.

In some aspects, the polyhydroxyalkanoate (PHA) particles in the composition can have a Dv50 particle size from about 1 μm to about 50 μm. In some aspects, the polyhydroxyalkanoate (PHA) particles in the composition have a Dv50 particle size from about 5 μm to about 20 μm. In some aspects, the polyhydroxyalkanoate (PHA) particles in the composition have a Dv50 particle size from about 8 μm to about 15 μm.

The particle size coefficient of variation (COV) is an additional approach for describing distribution width by normalizing the standard deviation through division by the mean. The particle size coefficient of variation can be measured using the formula:

Particle size coefficient of variation=(standard deviation/mean)*100.

The particle size coefficient of variation is provided as a percentage and does not have units. The COV is included in the laser diffraction software and is calculated automatically.

In some aspects, the composition of PHA particles can have a particle size coefficient of variation greater than 55%. In some aspects, the composition of PHA particles can have a particle size coefficient of variation greater than 50%. In some aspects, the composition of PHA particles can have a particle size coefficient of variation greater than 45%. In some aspects, the composition of PHA particles can have a particle size coefficient of variation greater than 40.5%. In some embodiments, the composition of PHA particles can have a particle size coefficient of variation of greater than about 40.5%, about 45%, about 50%, or about 55%.

In some aspects, the composition of PHA particles can comprise a compound of Formula I, a compound of Formula (II), a compound of Formula (III), a compound of Formula (IV), a compound of Formula (V), or a combination thereof. In some aspects, the composition of PHA particles can comprise a compound of Formula (I) and a compound of Formula (II). In some aspects, the composition of PHA particles can comprise a compound of Formula (I) and a compound of Formula (III). In some aspects, the composition of PHA particles can comprise a compound of Formula (I) and a compound of Formula (IV). In some aspects, the composition of PHA particles can comprise a compound of Formula (I) and a compound of Formula (V). In some aspects, the composition of PHA particles can comprise a compound of Formula (I) and a compound of Formula (VI).

In some aspects, the composition of PHA particles can further comprise one or more plasticizer selected from the group consisting of cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂₋₁₃ alkyl lactate, C₁₂₋₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂₋₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxystearate, trimethylpentanediol/adipic acid/glycerin crosspolymer, capryloyl glycerin/sebacic acid copolymer, brassica campestris/aleurites fordi oil copolymer, PPG-12/SMDI copolymer, polyisobutylene, polyisobutene 1200, polyhydroxystearic acid, and dicaprylyl/capryl sebacate, LEXOREZ 200 MB, VELLAPLEX MB, GLOSSAMER L6600, ADEKA NOL OU-1, LIPFEEL Natural MB, DERMOL ICSA, KESTER WAX K-60P, HALLGREEN CCS, citric acid, a citric acid derivative, glycerin ester, a glycerin ester derivative, adipic acid, an adipic acid derivative, phthalic acid, a phthalic acid derivative, and combinations thereof.

In some aspects, the citric acid derivative is acetyl tri-n-butyl citrate, acetyl triethyl citrate, tri-n-butyl citrate, triethyl citrate, or a combinations thereof. In some aspects, the glycerin derivative is glyceryl triacetate. In some aspects, the derivative of adipic acid is di(2-ethylhexyl) adipate, di(n-heptyl, n-nonyl) adipate, di(tridecyl) adipate, dicapryl adipate, diisobutyl adipate, diisodecyl adipate, diisononyl adipate, and combinations thereof. In some embodiments, the phthalic acid derivative is butyl benzyl phthalate, butyl octyl phthalate, di-n-butyl phthalate, di(2-ethylhexyl) phthalate, dicapryl phthalate, dicyclohexyl phthalate, diethyl phthalate, dihexyl phthalate, diisobutyl phthalate, diisodecyl phthalate, diisoheptyl phthalate, diisononyl phthalate, diisoctyl phthalate, dimethyl phthalate, ditridecyl phthalate, diundecyl phthalate, undecyl dodecyl phthalate, and combinations thereof. In some aspects, the composition of PHA particles can comprise one or more plasticizer selected from the group consisting of citric acid, a citric acid derivative, glycerin ester, a glycerin ester derivative, adipic acid, an adipic acid derivative, phthalic acid, a phthalic acid derivative, and combinations thereof.

In some aspects, the plasticizer can provide additional consistency and stability to the composition of PHA particles. Plasticizers can fix graininess that occurs when solids migrate and agglomerate. Plasticizers can also fix thermal stability issues by increasing melt point, without affecting the overall texture.

In some aspects, the composition of PHA particles can comprise a total weight of one or more plasticizer in a weight ratio from about 0.01% to about 80% of the total weight of the composition. In some aspects, the composition of PHA particles can comprise a total weight of one or more plasticizer in a weight ratio from about 0.01% to about 80%, from about 0.01% to about 60%, from about 0.01% to about 40%, from about 0.01% to about 20%, from about 0.01% to about 10%, from about 0.01% to about 5%, from about 0.01% to about 1%, from about 1% to about 80%, from about 1% to about 60%, from about 1% to about 40%, from about 1% to about 20%, from about 1% to about 10%, from about 1% to about 5%, from about 5% to about 80%, from about 5% to about 60%, from about 5% to about 40%, from about 5% to about 20%, from about 5% to about 10%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% of the total weight of the composition of PHA particles. In some aspects, the composition of PHA particles can comprise total weight of one or more plasticizer in a weight ratio of about 0.01%, about 1%, about 5%, about 10%, about 20%, about 40%, about 60%, or about 80% of the total weight of the composition.

### III. Methods of Preparing a Composition of PHA Particles

### 1. General Method

In some aspects, the present disclosure provides a method of preparing a composition of polyhydroxyalkanoate (PHA) particles comprising:
(a) mixing at least one polyhydroxyalkanoate (PHA) polymer and at least one additional polymer that is not a PHA polymer; and
(b1) melt-kneading the mixture of (a); or
(b2) heating the mixture of (a) for a time from about 1 minute to about 48 hours.

In some aspects, the present disclosure provides a method of preparing a composition of polyhydroxyalkanoate (PHA) particles comprising:
(a) mixing at least one PHA polymer and at least one additional polymer that is not a PHA polymer; and
(b1) melt-kneading the mixture of (a) at a temperature from about 40° C. to about 220° C.; or
(b2) heating the mixture of (a) at a temperature between about 40° C. to about 220° C. for a time of about 1 minute to about 48 hours.

In some aspects, the method of preparing a composition of PHA particles comprises one PHA polymer. In some aspects, the method of preparing a composition of PHA particles comprises two PHA polymers. In some aspects, the method of preparing a composition comprising PHA particles comprises three PHA polymers.

In some aspects, the method of preparing a composition of PHA particles comprises at least one additional polymer that is not a PHA polymer. In some aspects, the method of preparing a composition of PHA particles comprises one additional polymer. In some aspects, the method of preparing a composition of PHA particles comprises two additional polymers. In some aspects, the method of preparing a composition of PHA particles comprises three additional polymers.

In some aspects, the at least one additional polymer is selected from the group consisting of polyvinyl alcohol (PVA), polyethylene glycol (PEG), sodium polyacrylate, polyvinyl pyrrolidone, polypropylene oxide, polyglycerin, polyethylene oxide (PEO), vinyl acetate, modified starch, thermoplastic starch, methyl cellulose, ethyl cellulose, hydroxyethyl cellulose, and hydroxypropyl cellulose. In some aspects, the at least one additional polymer is selected from the group consisting of polyvinyl alcohol, polyethylene glycol and thermoplastic starch. In some aspects, the at least one additional polymer is selected from the group consisting of polyvinyl alcohol and thermoplastic starch. In some aspects, the at least one additional polymer is polyvinyl alcohol. In some aspects, the at least one additional polymer is thermoplastic starch.

In some aspects, the molar ratio of the total moles of at least one PHA polymer to the total moles of at least one additional polymer is from about 1:4 to about 2:3. In some aspects, the at least one additional polymer can be PVA. In some aspects, the molar ratio of the total moles of at least one PHA polymer to the total moles of PVA is from about 1:3 to about 1:1.5. In some aspects, the molar ratio of the total moles of at least one PHA polymer to the total moles of PVA is from about 1:2.5 to about 1:1.5. In some aspects, the molar ratio of the total moles of at least one PHA polymer to the total moles of PVA is from about 1:4, about 1:3, about 1:2.5, about 1:1.5, or about 2:3.

In some aspects, the method of preparing a composition comprising PHA particles further comprises mixing one or more plasticizer with the at least one PHA polymer and the at least one additional polymer. In some aspects, the one or more plasticizer is selected from the group consisting of cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂₋₁₃ alkyl lactate, C₁₂₋₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂₋₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxystearate, trimethylpentanediol/adipic acid/glycerin crosspolymer, capryloyl glycerin/sebacic acid copolymer, brassica campestris/aleurites fordi oil copolymer, PPG-12/SMDI copolymer, polyisobutylene, polyisobutene 1200, polyhydroxystearic acid, and dicaprylyl/capryl sebacate, LEXOREZ 200 MB, VELLAPLEX MB, GLOSSAMER L6600, ADEKA NOL OU-1, LIPFEEL Natural MB, DERMOL ICSA, KESTER WAX K-60P, HALLGREEN CCS, citric acid, a citric acid derivative, glycerin ester, a glycerin ester derivative, adipic acid, an adipic acid derivative, phthalic acid, a phthalic acid derivative, and combinations thereof. Mixing can be performed using a FlackTek Speedmixer.

In some aspects, the method of preparing a composition comprising PHA particles can comprise one or more plasticizer selected from the group consisting of citric acid, a citric acid derivative, glycerin ester, a glycerin ester derivative, adipic acid, an adipic acid derivative, phthalic acid, a phthalic acid derivative, and combinations thereof.

In some aspects, the mixing time for preparing a composition comprising PHA particles can be from about 1 minute to about 48 hours. In some aspects, the mixing time can be from about 1 minute to about 24 hours. In some aspects, the mixing time can be from about 2 minutes to about 48 hours. In some aspects, the mixing time can be from about 2 minutes to about 24 hours. In some aspects, the mixing time can be from about 30 minutes to about 12 hours. In some aspects, the mixing time can be from about 30 minutes to about 24 hours. In some aspects, the mixing time can be from about 1 hour to about 24 hours. In some aspects, the mixing time can be from about 1 minute to about 12 hours. In some aspects, the mixing time can be from about 1 minute to about 5 hours. In some aspects, the mixing time can be from about 1 minute to about 1 hour. In some aspects, the mixing time can be about 1 minute, about 1.5 minutes, about 2 minutes, about 2.5 minutes, about 3 minutes, about 3.5 minutes, about 6 minutes, about 12 minutes, about 30 minutes, about 60 minutes, about 2 hours, about 4 hours, about 6 hours, about 12 hours, about 24 hours, or about 48 hours. In some aspects, the mixing time can be about 1 minute. In some aspects, the mixing time can be about 2 minutes. In some aspects, the mixing time can be about 3 minutes. In some aspects, the mixing time can be about 3.5 minutes.

In some aspects, the mixing time for preparing a composition of PHA particles can occur at a speed from about 5 rpm to about 2000 rpm. In some aspects, the mixing can occur at a speed from about 10 rpm to about 900 rpm. In some aspects, the mixing can occur at a speed from about 10 rpm to about 800 rpm. In some aspects, the mixing can occur at a speed from about 50 rpm to about 700 rpm. In some aspects, the mixing can occur at a speed of about 5 rpm, about 10 rpm, about 15 rpm, about 20 rpm, about 25 rpm, about 30 rpm, about 35 rpm, about 40 rpm, about 45 rpm, about 50 rpm, about 55 rpm, about 60 rpm, about 65 rpm, about 70 rpm, about 75 rpm, about 80 rpm, about 85 rpm, about 90 rpm, about 95 rpm, about 100 rpm, about 150 rpm, about 200 rpm, about 250 rpm, about 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, about 950 rpm, or about 1000 rpm. In some aspects, the mixing can occur at a speed of about 600 rpm. In some aspects, the mixing can occur at a speed of about 700 rpm. In some aspects, the mixing can occur at a speed of about 900 rpm.

### 2. Extrusion Method

In some aspects, the present disclosure provides a method of preparing a composition of polyhydroxyalkanoate (PHA) particles comprising:
(a) mixing at least one PHA polymer and at least one additional polymer that is not a PHA polymer, in an extruder;
(b) melt-kneading the mixture of (a) for a time from about 1 minute to about 48 hours; and
(c) extruding the mixture of (b).

In some aspects, the present disclosure provides a method of preparing a composition of PHA particles comprising:
(a) mixing at least one PHA polymer and at least one additional polymer that is not a PHA polymer, in an extruder; and
(b) melt-kneading the mixture of (a) at a temperature from about 40° C. to about 220° C. for a time from about 1 minute to about 48 hours; and
(c) extruding the mixture of (b).

In some aspects, the PHA particles are prepared using a closed system. In some aspects, the closed system comprises a N₂-flushed reactor equipped with a heating mantle, overhead stirrer, reflux condenser, addition funnel, and thermocouple.

In some aspects, the method of preparing a composition of PHA particles further comprises addition of water to wash the extruded dispersion of particles.

In some aspects, the at least one additional polymer is selected from the group consisting of polyvinyl alcohol (PVA), polyethylene glycol (PEG), sodium polyacrylate, polyvinyl pyrrolidone, polypropylene oxide, polyglycerin, polyethylene oxide (PEO), vinyl acetate, modified starch, thermoplastic starch, methyl cellulose, ethyl cellulose, hydroxyethyl cellulose, and hydroxypropyl cellulose. In some aspects, the at least one additional polymer is selected from the group consisting of polyvinyl alcohol, polyethylene glycol and thermoplastic starch. In some aspects, the at least one additional polymer is selected from the group consisting of polyvinyl alcohol and thermoplastic starch. In some aspects, the at least one additional polymer is polyvinyl alcohol. In some aspects, the at least one additional polymer is thermoplastic starch.

In some aspects, the weight average molecular weight (M_{W}) of the at least one additional polymer is from about 500 g/mol to about 100,000 g/mol. In some aspects, the weight average molecular weight of the at least one PHA polymer is from about 500 g/mol to about 100,000 g/mol, from about 10,000 g/mol to about 50,000 g/mol, from about 10,000 g/mol to about 100,000 g/mol, or from about 50,000 g/mol to about 100,000 g/mol.

In some aspects, the molar ratio of the total moles of the at least one PHA polymer to the total moles of the at least one additional polymer is from about 1:4 to about 2:3. In some aspects, the molar ratio of the total moles of the at least one PHA to the total moles of the at least one PVA is from about 1:3 to about 1:1.5. In some aspects, the molar ratio of the total moles of the at least one PHA to the total moles of the at least one PVA is from about 1:2.5 to about 1:1.5. In some aspects, the molar ratio of the total moles of the at least one PHA to the total moles of the at least one PVA is from about 1:4, about 1:3, about 1:2.5, about 1:1.5, or about 2:3.

In some aspects, the method of preparing a composition of PHA particles can comprise mixing one or more plasticizer with the at least one PHA polymer and the at least one additional polymer. In some aspects, the one or more plasticizer is selected from the group consisting of cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂₋₁₃ alkyl lactate, C₁₂₋₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂₋₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxystearate, trimethylpentanediol/adipic acid/glycerin crosspolymer, capryloyl glycerin/sebacic acid copolymer, brassica campestris/aleurites fordi oil copolymer, PPG-12/SMDI copolymer, polyisobutylene, polyisobutene 1200, polyhydroxystearic acid, and dicaprylyl/capryl sebacate, LEXOREZ 200 MB, VELLAPLEX MB, GLOSSAMER L6600, ADEKA NOL OU-1, LIPFEEL Natural MB, DERMOL ICSA, KESTER WAX K-60P, HALLGREEN CCS, citric acid, a citric acid derivative, glycerin ester, a glycerin ester derivative, adipic acid, an adipic acid derivative, phthalic acid, a phthalic acid derivative, and combinations thereof.

In some aspects, the one or more plasticizer is selected from the group consisting of citric acid, a citric acid derivative, glycerin ester, a glycerin ester derivative, adipic acid, an adipic acid derivative, phthalic acid, a phthalic acid derivative, and combinations thereof.

In some aspects, the mixing time can be from about 1 minute to about 48 hours. In some aspects, the mixing time can be from about 1 minute to about 24 hours. In some aspects, the mixing time can be from about 2 minutes to about 48 hours. In some aspects, the mixing time can be from about 2 minutes to about 24 hours. In some aspects, the mixing time can be from about 30 minutes to about 12 hours. In some aspects, the mixing time can be from about 30 minutes to about 24 hours. In some aspects, the mixing time can be from about 1 hour to about 24 hours. In some aspects, the mixing time can be from about 1 minute to about 12 hours. In some aspects, the mixing time can be from about 1 minute to about 5 hours. In some aspects, the mixing time can be from about 1 minute to about 1 hour. In some aspects, the mixing time can be about 1 minute, about 1.5 minutes, about 2 minutes, about 2.5 minutes, about 3 minutes, about 3.5 minutes, about 6 minutes, about 12 minutes, about 30 minutes, about 60 minutes, about 2 hours, about 4 hours, about 6 hours, about 12 hours, about 24 hours, or about 48 hours. In some aspects, the mixing time can be about 1 minute. In some aspects, the mixing time can be about 2 minutes. In some aspects, the mixing time can be about 3 minutes. In some aspects, the mixing time can be about 3.5 minutes.

In some aspects, the mixing can occur at a speed from about 5 rpm to about 2000 rpm. In some aspects, the mixing can occur at a speed from about 10 rpm to about 900 rpm. In some aspects, the mixing can occur at a speed from about 10 rpm to about 800 rpm. In some aspects, the mixing can occur at a speed from about 50 rpm to about 700 rpm. In some aspects, the mixing can occur at a speed of about 5 rpm, about 10 rpm, about 15 rpm, about 20 rpm, about 25 rpm, about 30 rpm, about 35 rpm, about 40 rpm, about 45 rpm, about 50 rpm, about 55 rpm, about 60 rpm, about 65 rpm, about 70 rpm, about 75 rpm, about 80 rpm, about 85 rpm, about 90 rpm, about 95 rpm, about 100 rpm, about 150 rpm, about 200 rpm, about 250 rpm, about 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, about 950 rpm, or about 1000 rpm. In some aspects, the mixing can occur at a speed of about 600 rpm. In some aspects, the mixing can occur at a speed of about 700 rpm. In some aspects, the mixing can occur at a speed of about 900 rpm.

In some aspects, the melt-knead time can be from about 1 minute to about 48 hours. In some aspects, the melt-knead time can be from about 1 minute to about 24 hours. In some aspects, the melt-knead time can be from about 2 minutes to about 48 hours. In some aspects, the melt-knead time can be from about 2 minutes to about 24 hours. In some aspects, the melt-knead time can be from about 30 minutes to about 12 hours. In some aspects, the melt-knead time can be from about 30 minutes to about 24 hours. In some aspects, the melt-knead time can be from about 1 hour to about 24 hours. In some aspects, the melt-knead time can be from about 1 minute to about 12 hours. In some aspects, the melt-knead time can be from about 1 minute to about 5 hours. In some aspects, the melt-knead time can be from about 1 minute to about 1 hour. In some aspects, the melt-knead time can be about 1 minute, about 1.5 minutes, about 2 minutes, about 2.5 minutes, about 6 minutes, about 12 minutes, about 30 minutes, about 60 minutes, about 2 hours, about 5 hours, about 10 hours, about 24 hours, or about 48 hours. In some aspects, the melt-knead time can be about 1.5 minutes. In some aspects, the melt-knead time can be about 2 minutes. In some aspects, the melt-knead time can be about 2.5 minutes.

In some aspects, the melt-kneading can occur at a temperature from about 40° C. to about 220° C. In some aspects, the melt-kneading can occur at a temperature from about 50° C. to about 210° C. or, about 55° C. to about 200° C. In some aspects, the melt- kneading can occur at a temperature from about 100° C. to about 220° C. or, about 150° C. to about 220° C. In some aspects, the melt-kneading can occur at a temperature of about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., about 200° C., about 205° C., about 210° C., about 215° C., or about 220° C. In some aspects, the melt-kneading can occur at a temperature of about 50° C. In some aspects, the melt-kneading can occur at a temperature of about 55° C. In some aspects, the melt-kneading can occur at a temperature of about 190° C. In some aspects, the melt-kneading can occur at a temperature of about 200° C.

In some aspects, the melt-kneading can occur at a speed from about 5 rpm to about 1000 rpm. In some aspects, the melt-kneading can occur at a speed from about 10 rpm to about 900 rpm. In some aspects, the melt-kneading can occur at a speed from about 10 rpm to about 800 rpm. In some aspects, the melt-kneading can occur at a speed from about 50 rpm to about 700 rpm. In some aspects, the melt-kneading can occur at a speed of about 5 rpm, about 10 rpm, about 15 rpm, about 20 rpm, about 25 rpm, about 30 rpm, about 35 rpm, about 40 rpm, about 45 rpm, about 50 rpm, about 55 rpm, about 60 rpm, about 65 rpm, about 70 rpm, about 75 rpm, about 80 rpm, about 85 rpm, about 90 rpm, about 95 rpm, about 100 rpm, about 150 rpm, about 200 rpm, about 250 rpm, about 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, about 950 rpm, or about 1000 rpm. In some aspects, the melt-kneading can occur at a speed of about 600 rpm. In some aspects, the melt-kneading can occur at a speed of about 700 rpm. In some aspects, the melt-kneading can occur at a speed of about 900 rpm.

### 3. Solvent Evaporation Method

In some aspects, the present disclosure provides a method of preparing a composition of polyhydroxyalkanoate (PHA) particles comprising:
(a) mixing at least one polyhydroxyalkanoate (PHA) polymer and at least one additional polymer that is not a PHA polymer; and
(b) heating the mixture of (a) for a time from about 1 minute to about 48 hours.

In some aspects, the present disclosure is directed to a method of preparing a composition of PHA particles comprising:
(a) mixing at least one PHA polymer, at least one additional polymer that is not a PHA polymer, and at least one organic solvent; and
(b) heating the mixture of (a) at a temperature between about 40° C. to about 220° C. for a time of about 1 minute to about 48 hours.

In some embodiments, the method of preparing a composition of PHA particles further comprises:
(c) adding water to the mixture of (b) to form an emulsion;
(d) mixing the emulsion of (c);
(e) heating the mixture of (d) for a time of from about 1 hour to about 48 hours; and
(f) filtering the mixture of (e) to obtain the composition of PHA particles.

In some aspects, the composition of PHA particles are prepared using a closed system. In some aspects, the closed system comprises a N₂-flushed reactor equipped with a heating mantle, overhead stirrer, reflux condenser, addition funnel, and thermocouple.

In some aspects, the method further comprises addition of water to wash the particles.

In some aspects, the at least one additional polymer is selected from the group consisting of polyvinyl alcohol (PVA), polyethylene glycol (PEG), sodium polyacrylate, polyvinyl pyrrolidone, polypropylene oxide, polyglycerin, polyethylene oxide (PEO), vinyl acetate, modified starch, thermoplastic starch, methyl cellulose, ethyl cellulose, hydroxyethyl cellulose, and hydroxypropyl cellulose. In some aspects, the at least one additional polymer is selected from the group consisting of polyvinyl alcohol, polyethylene glycol and thermoplastic starch. In some aspects, the at least one additional polymer is selected from the group consisting of polyvinyl alcohol and thermoplastic starch. In some aspects, the at least one additional polymer is polyvinyl alcohol. In some aspects, the at least one additional polymer is thermoplastic starch.

In some aspects, the weight average molecular weight (M_{W}) of the at least one additional polymer is from about 500 g/mol to about 100,000 g/mol. In some aspects, the weight average molecular weight of the at least one PHA polymer is from about 500 g/mol to about 100,000 g/mol, from about 10,000 g/mol to about 50,000 g/mol, from about 10,000 g/mol to about 100,000 g/mol, or from about 50,000 g/mol to about 100,000 g/mol. In some aspects, the molar ratio of the total moles of at least one PHA polymer to the moles of at least one additional polymer is from about 1:4 to about 2:3. In some aspects, the molar ratio of the moles of at least one PHA to the total moles of PVA is from about 1:3 to about 1:1.5. In some aspects, the molar ratio of the total moles of at least one PHA to the total moles of PVA is from about 1:2.5 to about 1:1.5. In some aspects, the molar ratio of the total moles of at least one PHA to the total moles of PVA is from about 1:4, about 1:3, about 1:2.5, about 1:1.5, or about 2:3.

In some aspects, the mixing in (a) further comprises one or or more plasticizer selected from the group consisting of cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂₋₁₃ alkyl lactate, C₁₂₋₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂₋₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxystearate, trimethylpentanediol/adipic acid/glycerin crosspolymer, capryloyl glycerin/sebacic acid copolymer, brassica campestris/aleurites fordi oil copolymer, PPG-12/SMDI copolymer, polyisobutylene, polyisobutene 1200, polyhydroxystearic acid, and dicaprylyl/capryl sebacate, LEXOREZ 200 MB, VELLAPLEX MB, GLOSSAMER L6600, ADEKA NOL OU-1, LIPFEEL Natural MB, DERMOL ICSA, KESTER WAX K-60P, HALLGREEN CCS, citric acid, a citric acid derivative, glycerin ester, a glycerin ester derivative, adipic acid, an adipic acid derivative, phthalic acid, a phthalic acid derivative, and combinations thereof.

In some aspects, the mixing in (a) further comprises one or more plasticizer selected from the group consisting of citric acid, a citric acid derivative, glycerin ester, a glycerin ester derivative, adipic acid, an adipic acid derivative, phthalic acid, a phthalic acid derivative, and combinations thereof.

In some aspects, the at least one organic solvent is selected from the group consisting of chloroform, ethyl acetate, dichloromethane, dimethylformamide, dimethyl sulfoxide, N-methyl-2-pyrrolidone or combinations thereof. In some aspects, the at least one organic solvent is chloroform.

In some aspects, the mixing time in (a) can be from about 1 minute to about 48 hours. In some aspects, the mixing time can be from about 1 minute to about 24 hours. In some aspects, the mixing time can be from about 2 minutes to about 48 hours. In some aspects, the mixing time can be from about 2 minutes to about 24 hours. In some aspects, the mixing time can be from about 30 minutes to about 12 hours. In some aspects, the mixing time can be from about 30 minutes to about 24 hours. In some aspects, the mixing time can be from about 1 hour to about 24 hours. In some aspects, the mixing time can be from about 1 minute to about 12 hours. In some aspects, the mixing time can be from about 1 minute to about 5 hours. In some aspects, the mixing time can be from about 1 minute to about 1 hour. In some aspects, the mixing time can be about 1 minute, about 1.5 minutes, about 2 minutes, about 2.5 minutes, about 3 minutes, about 3.5 minutes, about 4 minutes, about 4.5 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 25 minutes, about 30 minutes, about 35 minutes, about 40 minutes, about 45 minutes, about 50 minutes, about 55 minutes, about 60 minutes, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 42 hours, about 42 hours, about 43 hours, about 44 hours, about 45 hours, about 46 hours, about 47 hours, or about 48 hours. In some aspects, the mixing time can be about 1 minute. In some aspects, the mixing time can be about 2 minutes. In some aspects, the mixing time can be about 3 minutes. In some aspects, the mixing time can be about 3.5 minutes.

In some aspects, the mixing in (a) can occur at a speed from about 5 rpm to about 1000 rpm. In some aspects, the mixing can occur at a speed from about 10 rpm to about 900 rpm. In some aspects, the mixing can occur at a speed from about 10 rpm to about 800 rpm. In some aspects, the mixing can occur at a speed from about 50 rpm to about 700 rpm. In some aspects, the mixing can occur at a speed of about 5 rpm, about 10 rpm, about 15 rpm, about 20 rpm, about 25 rpm, about 30 rpm, about 35 rpm, about 40 rpm, about 45 rpm, about 50 rpm, about 55 rpm, about 60 rpm, about 65 rpm, about 70 rpm, about 75 rpm, about 80 rpm, about 85 rpm, about 90 rpm, about 95 rpm, about 100 rpm, about 150 rpm, about 200 rpm, about 250 rpm, about 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, about 950 rpm, or about 1000 rpm. In some aspects, the mixing can occur at a speed of about 600 rpm. In some aspects, the mixing can occur at a speed of about 700 rpm. In some aspects, the mixing can occur at a speed of about 900 rpm.

In some aspects, the heating in (b) can occur at a temperature from about 40° C. to about 220° C. In some aspects, the method can occur at a temperature from about 50° C. to about 210° C. or, about 55° C. to about 200° C. In some aspects, the method can occur at a temperature from about 100° C. to about 220° C. or, about 150° C. to about 220° C. In some aspects, the method can occur at a temperature of about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., about 200° C., about 205° C., about 210° C., about 215° C., or about 220° C. In some aspects, the method can occur at a temperature of about 50° C. In some aspects, the method can occur at a temperature of about 55° C. In some aspects, the method can occur at a temperature of about 190° C. In some aspects, the method can occur at a temperature of about 200° C.

### IV. Personal Care Composition and Personal Care Formulation

In some aspects, the personal care composition comprising a composition of PHA particles can further comprise at least one additional component selected from the group consisting of water, aqueous solvent (e.g. alcohol or other water compatible solvent), non-aqueous solvent, emollients, humectants, oils (polar and non-polar), conditioning agents, emulsifiers, surfactants, thickeners, stiffening agents, medicaments, fragrances, preservatives, deodorant components, antiperspirant compounds, skin protecting agents, pigments, particulates, dyes, sunscreens and combinations thereof.

In some aspects, the personal care composition comprising a composition of PHA particles can further comprise at least one additional component selected from the group consisting of pigments, particulates, dyes, and combinations thereof.

In some aspects, the pigments, particulates, and dyes are selected from the group consisting of iron oxides, zinc oxides, titanium dioxides, mica, silica, tapioca starch, rice starch, cellulose, cellulose acetate, polymethylsilsesquioxane, mica, and combinations thereof.

In some aspects, the total weight of the at least one additional component can comprise from about 0.01% to about 80%, from about 0.01% to about 60%, from about 0.01% to about 40%, from about 0.01% to about 20%, from about 0.01% to about 10%, from about 0.01% to about 5%, from about 0.01% to about 1%, from about 1% to about 80%, from about 1% to about 60%, from about 1% to about 40%, from about 1% to about 20%, from about 1% to about 10%, from about 1% to about 5%, from about 5% to about 80%, from about 5% to about 60%, from about 5% to about 40%, from about 5% to about 20%, from about 5% to about 10%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% of the total weight of the composition. In some aspects, the personal care compositions can comprise a total weight of the at least one additional component in a weight ratio of about 0.01%, about 1%, about 5%, about 10%, about 20%, about 30%, about 40%, about 50%, about 60%, about 70%, or about 80% of the total weight of the personal care composition.

In some aspects, a personal care formulation is prepared by combining a personal care composition or a composition comprising PHA particles with at least one solvent to form a solid, gel, a paste, or a powder. In some aspects, the personal care formulation is a solid. In some aspects, the personal care formulation is a gel. In some aspects, the personal care formulation is a paste. In some aspects, the personal care formulation is a powder.

In some aspects, a personal care formulation can be prepared by shearing the composition comprising PHA particles, as described herein, with a solvent, as described herein, to form a sheared gel. In some aspects, a personal care formulation can be prepared by combining a personal care composition or a composition comprising PHA particles, as described herein, with a solvent thereby forming a mixture and shearing the mixture.

In some aspects, the personal care formulation comprising PHA particles can be crumbled to form a powder.

In some aspects, the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

### V. Composition of PHA Particles Prepared by Process

The present disclosure provides a composition comprising polyhydroxyalkanoate (PHA) particles, wherein the PHA particles are prepared by mixing (i) at least one polyhydroxyalkanoate (PHA) polymer; and (ii) at least one additional polymer that is not a PHA polymer.

### Examples

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Preparation of PHB Particles in PVA

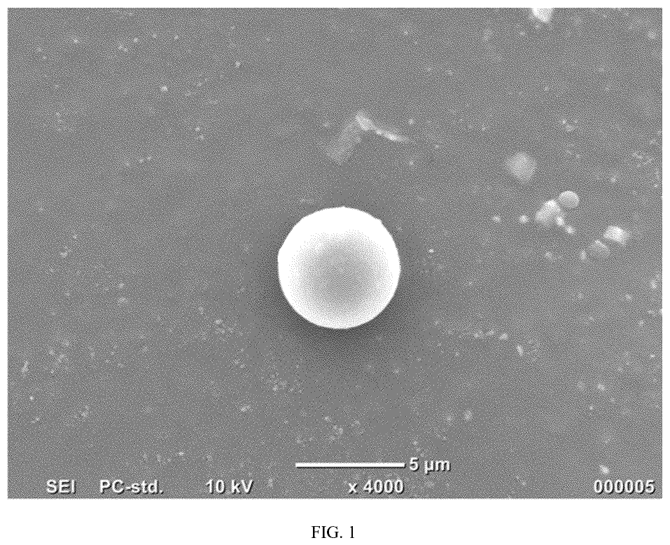

Polyhydroxybutyrate (PHB) (Tianan ENMAT® Y3000P pellets (M_{W}=280,000 g/mol)) and polyvinyl alcohol (PVA) (Density=0.6-0.9 mg/cm3) in a ratio of 30:70 weight were mixed together. 10 g of the mixture was loaded into a microextruder (Xplore 12 cc, twin-screw) and melt-kneaded for a time between 1 minute and 1 hour at a temperature between 150° C. and 250° C. The mixture was then extruded at a speed of from about 10 rpm to about 100 rpm at a temperature between 150° C. and 250° C. in the form of a solid dispersion. The collected solid dispersion of polymer material was washed several times in distilled water. Each wash was performed using 90° C. water with stirring for 2 hours. The solid polymer was filtered and dried at room temperature. Formation of spherical and smooth non-porous PHB particles with a particle volume median diameter of about 5 μm was confirmed by scanning electron microscopy (SEM) as shown in FIG. 1.

### Example 2: Preparation of PHB Particles in Glycerin

PHB (Tianan ENMAT Y3000P pellets (M_{W} 280,000 g/mol)) and glycerin in a ratio of 90:10 by weight were mixed together. To the mixture was added PVA in a ratio of 30:70 by weight. The mixture was loaded into a microextruder (Xplore 12 cc, twin-screw) and melt-kneaded for a time between about 1 minute and 1 hour at temperature between about 150° C. and about 250° C., then extruded at a speed of about 10 rpm to about 100 rpm at a temperature between about 150° C. and about 250° C. in the form of a solid dispersion. The collected solid dispersion of polymer material was washed several times in distilled water. Each wash was performed using 90° C. water with stirring for 2 hours. The solid polymer was filtered and dried at room temperature.

### Example 3: Preparation of PHB Particles in PVA

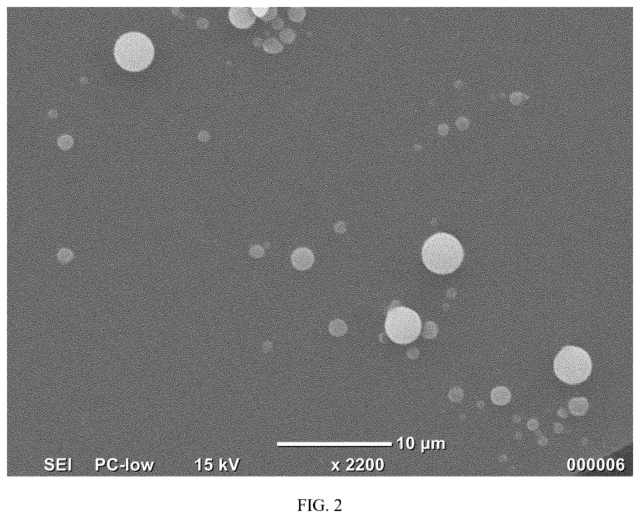

Polyhydroxybutyrate (PHB) (Tianan ENMAT® Y3000P pellets (M_{W}=280,000 g/mol)) and polyvinyl alcohol (PVA) (Density=0.6-0.9 mg/cm3) in a ratio of 30:70 weight were mixed together. 500 grams of the mixture was loaded into an extruder (Coperion ZSK 18, 18 mm twin extruder) and melt-kneaded for a time between about 1 minute and 1 hour at a temperature between about 150° C. and about 250° C., then extruded at a speed of about 10 rpm to about 100 rpm at a temperature between about 150° C. and about 250° C. in the form of a solid dispersion. The collected solid dispersion of polymer material was washed several times in distilled water. Each wash was performed using 90° C. water with stirring for 2 hours. The solid polymer was filtered and dried at room temperature. Formation of spherical and smooth non-porous PHB particles was confirmed by scanning electron microscopy (SEM) as shown in FIG. 2.

### Example 4: Preparation of PHBV Particles

50 grams of a 2.25% polyvinyl alcohol (PVA) solution was heated to 55° C. To the PVA solution was added 10 grams of a 1.34% poly(3-hydroxybutyrate-co-3-hydroxyvalerate) (PHBV) (Tianan ENMAT® Y1000 powder (M_{W}=240,000 g/mol)) solution in chloroform. The mixture was stirred at a constant speed between about 400 rpm and about 1000 rpm, at a temperature of between about 40° C. and about 100° C., and for a time of about 1 minute and about 1 hour. 800 mL of deionized water heated to between about 40° C. and about 100° C. was added to the mixture, and the emulsion was stirred at a constant speed between about 400 rpm and about 1000 rpm for a time of about 30 minutes and 3 hours. The mixture was centrifuged at 4500 rpm for 10 minutes and the supernatant was collected and filtered to obtain spherical and smooth particles. The particles were dried at room temperature overnight.

### Comparison of Prepared PHB Particles, Prepared PHBV Particles, PHB Starting Material, and PHBV Starting Material

Sphericity, solidity, and particle size measurements for the prepared PHB particles from Example 1, for the prepared PHBV particles from Example 4, for the PHB pellets used as a starting material in Example 1, and for the PHBV powder used as a starting material in Example 4 are provided in TABLE 1. As shown in TABLE 1, the sphericity, solidity, and particle size differed greatly for the PHB and PHBV particles subjected to the conditions of Example 1 and Example 4, respectively, as compared to the commercially available PHB and PHBV used as starting materials. As discussed herein, in order to achieve the necessary texture and optical effects, PHA particles must be spherical, have a smooth surface that is non-porous, and have a diameter of between 1 μm and 30 μm. As shown in TABLE 1, the PHB and PHBV starting materials were not spherical, had low porosity, and the individual particles had a particle size outside the desired range. After the PHB and PHBV starting materials were subjected to the conditions of Examples 1 and 4, respectively, the resultant particles were found to be spherical, porous, and a particle volume median diameter between 5 μm and 20 μm (i.e., within the desired range). As discussed herein, the sphericity, porosity/solidity, and particle size of PHA particles are critical properties for providing successful performance and optimal sensory when used in a beauty and/or cosmetic product.

TABLE 1
Characterization of PHB Particles, PHBV Particles,
PHB Starting Material, and PHBV Starting Material

| | PHB starting | PHBV starting | Exam- | Exam- |
|---|---|---|---|---|
| | material*¹ | material*² | ple 1 | ple 4 |
| Sphericity | <0.5 | Shapeless large aggregates | 1.00 | 1.00 |
| | | of nanoparticles of | | |
| | | sphericity <0.5 | | |
| Porosity/ | Pellets: 0.9 | Individual particles: | 100% | 100% |
| solidity | | 1.00 | | |
| Particle | Pellets: 3-5 | Large aggregates: | 5-20 μm | 5-20 μm |
| size | mm | 5-10 μm; | | |
| | | Individual particles: | | |
| | | <1 μm | | |
| COV | N/A | N/A | 47% | 45% |

*¹= PHB Y3000P pellets obtained from Tianan ENMAT ® (Mw = 280,000 g/mol)
*²= PHBV Y1000 powder obtained from Tianan ENMAT ® (Mw = 240,000 g/mol)

### Comparison of Prepared PHB Particles, Prepared PHBV Particles, PHB Starting Material, and PHBV Starting Material

Morphology, porosity, particle size distribution, monodispersity, and biodegradability was measured for the prepared PHB particles from Example 1, for the prepared PHBV particles from Example 4, and for the PHBV powder used as a starting material in Example 4 are provided in TABLE 2. As shown in TABLE 2, the morphology, particle size, and monodispersity differed greatly for the PHB and PHBV particles subjected to the conditions of Example 1 and Example 4, respectively, as compared to the commercially available PHBV used as a starting material. As discussed herein, in order to achieve the necessary texture and optical effects, PHA particles must be spherical, have a smooth surface that is non-porous, and have a particle volume median diameter of between 1 μm and 30 μm. When particles lack these properties it results in products with increased coarseness, decreased spreadability, decreased glide, and a worsened overall sensory feel.

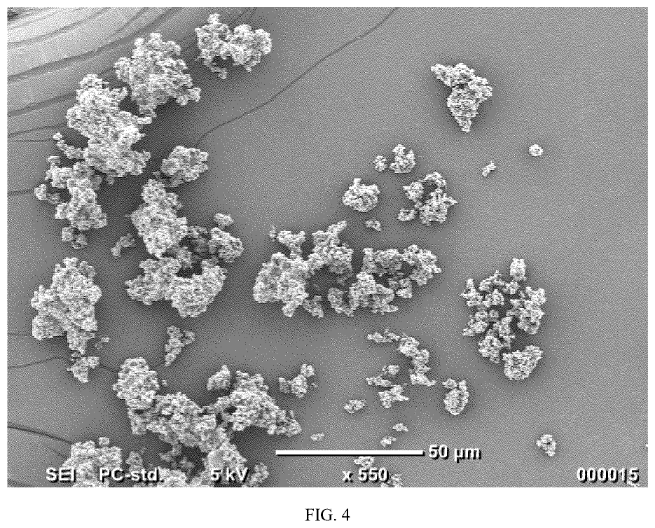

Even though the commercially available PHBV particles were spherical and non-porous, they did not meet the particle size requirements and were not monodisperse. The commercially available PHBV particles fall under the class of nanoparticles and as such, tend to clump together in large aggregates rather than remaining monodisperse, as observed in the SEM image in FIG. 4. Both of the methods of making PHA particles disclosed in Example 1 and Example 4 meet the requirements for spherical, smooth, non-porous, monodisperse, and having a particle volume median diameter of between 1 μm and 30 μm-requirements that are necessary to provide the optimal properties desired in personal care compositions and personal care formulations.

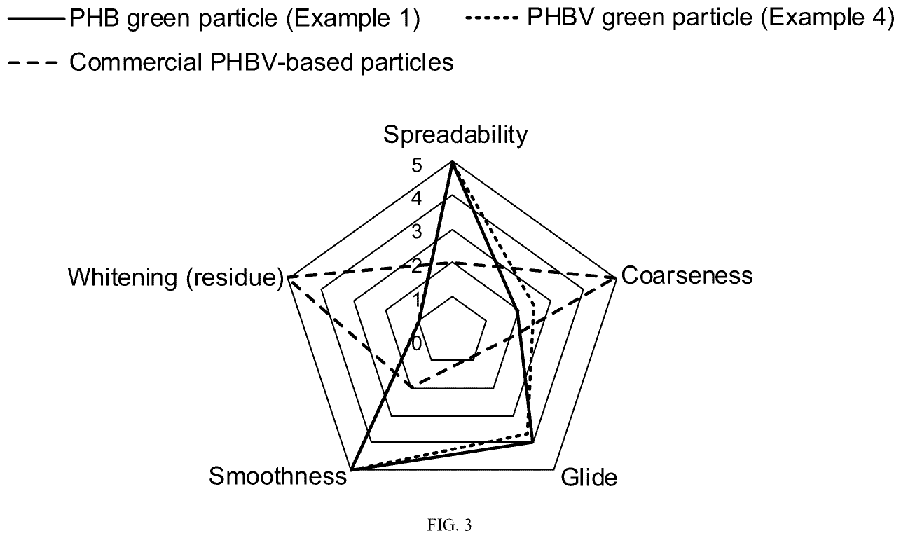

Additional sensory properties for the prepared PHB particles from Example 1, for the prepared PHBV particles from Example 4, and for the commercially available PHBV powder used as a starting material in Example 4 are shown in the spider chart in FIG. 3. In particular, the spreadability, the whitening, the smoothness, the glide, and the coarseness for these materials were evaluated. As shown in FIG. 3, the commercially available PHBV powder provided a spreadability of 1/5, a whiteness of 5/5, a smoothness of 1/5, a glide of 1/5, and a coarseness of 5/5. The prepared PHB particles from Example 1 provided a spreadability of 5/5, a whiteness of 0/5, a smoothness of 5/5, a glide of 4/5, and a coarseness of 1.5/5. And, the prepared PHBV particles from Example 3 provided a spreadability of 5/5, a whiteness of 0/5, a smoothness of 5/5, a glide of 4/5, and a coarseness of 1/5. Thus, the prepared PHB and PHBV provide sensory properties that are desirable in personal care compositions and personal care formulations.

TABLE 2
Characterization of PHB Particles, PHBV
Particles, and PHBV Starting Material.

| | PHB Particles | PHBV Particles | Commercial PHBV |
|---|---|---|---|
| | of Example 1 | of Example 4 | Particles*³ |
| Morphology | Spherical, | Spherical, | Spherical, |
| | Smooth | Smooth | Coarse Lμmps |
| Porosity | Non-Porous | Non-Porous | Non-Porous |
| Particle Volume | 5-20 μm | 5-20 μm | Large Aggregates, |
| Median Diameter | | | individual Particles |
| Range | | | are ≤1 μm |
| Monodisperse | Yes | Yes | No |
| Biodegradable | Yes | Yes | Yes |

*³= PHBV Y1000 powder obtained from Tianan ENMAT ® (Mw = 240,000 g/mol)

### Comparison of Cosmetic Formulations Using Prepared PHB Particles, Prepared PHBV Particles, and Commercially Available PHBV Particles

Cosmetic formulations were prepared using the prepared PHB particles of Example 1, the prepared PHBV particles of Example 4, and commercially available PHBV particles. The ingredients used to prepare the cosmetic formulations are provided in TABLE 3. The cosmetic formulations were prepared by (1) combining the ingredients and mixing until uniform; and (2) adding fragrance as desired and mixing until uniform.

TABLE 3
Cosmetic Formulations Using Prepared PHB Particles, Prepared
PHBV Particles, and Commercially Available PHBV.

| | Formulation | Formulation | Formulation |
|---|---|---|---|
| | Comprising | Comprising | Comprising |
| | PHB | PHBV | Commercial |
| | Particles of | Particles of | PHBV |
| Ingredient | Example 1 | Example 4 | Particles*⁴ |
| Example 1 PHB | 20% | — | — |
| Particles | | | |
| Example 4 PHBV | — | 20% | — |
| Particles | | | |
| Commercial PHBV | — | — | 20% |
| Particles | | | |
| Crosslinked | 60% | 60% | 60% |
| Polymer | | | |
| Solvent | 20% | 20% | 20% |

*⁴= PHBV Y1000 powder obtained from Tianan ENMAT ® (Mw = 240,000 g/mol)

Cosmetic formulations prepared with the prepared PHV particles of Example 1 and the prepared PHBV particles of Example 4 provide an enhanced sensory feel including increased spreadability and smoothness of the final product with an absence of the whitening effect. These attributes are lacking in the cosmetic formulation prepared from commercially available PHBV particles which show increased coarseness, decreased spreadability, and a whitening effect when applied to the skin.

### Example 5: Additional Cosmetic Formulations

### Formulation 1: Powder Foundation

Powder foundations were prepared with the ingredients shown in TABLE 4 using the prepared PHB particles of Example 1. In the preparation method, ingredients of Phase A and Phase B were first mixed in an IKA Lab Mixer. Then premixed ingredients of Phase C were added. The resulting mixture was mixed until uniform using the IKA Lab Mixer. Finally the mixture was compression molded into a metal plate.

TABLE 4
Powder Foundation Formulation

| | | Amount |
|---|---|---|
| Phase | Ingredient | (Wt. %) |
| A | Spherical PHB particles from Example 1 | 10 |
| | Polymethylhydrosiloxane treated Talc | 22 |
| | Polymethylhydrosiloxane treated Mica | 33 |
| | Polymethylhydrosiloxane treated Titanium Dioxide | 11.5 |
| | Polymethylhydrosiloxane treated Zinc Oxide | 3 |
| | Boron Nitride Powder | 4 |
| | Microsphere -M-305 (Polymethylmethacrylate Particles) | 1 |
| | (Matsumoto Yushi-Seiyaku Co. Ltd) | |
| | Magnesium Stearate | 2 |
| B | Polymethylhydrosiloxane treated Iron Oxides (CI 77492 | 2.2 |
| | (Yellow Iron Oxide)) | |
| | Polymethylhydrosiloxane treated Iron Oxides (CI 77491 | 0.54 |
| | (Red Iron Oxide)) | |
| | Polymethylhydrosiloxane treated Iron Oxides (CI 77499 | 0.36 |
| | (Black Iron Oxide)) | |
| C | Polydimethylsiloxane trimethylsiloxysilicate | 2 |
| | (Momentive SS4267) | |
| | Pentylene Glycol | 2 |
| | Silsoft B 3820 (Momentive) (Isododecane and | 1 |
| | Dimethicone Blend) | |
| | Caprylic/Capric Triglyceride | 1.6 |
| | Sorbitan Sesquiisostearate | 0.5 |
| | Diisostearyl Malate | 0.5 |
| | Ethylhexyl Methoxycinnamate | 2 |
| | Tocopheryl Acetate | 0.5 |
| | Phenoxylethanol | 0.3 |

### Formulation 2: Powder Formulation

Powder formulations were prepared with the ingredients in TABLE 5 using the prepared PHBV particles of Example 4. The ingredients were mixed in a 100 gm speedmixer container and the powders were mixed for 5 minutes to obtain a uniformly mixed loose powder formulation.

TABLE 5
Powder Formulation

| Ingredient | | Amount |
|---|---|---|
| No. | Ingredient | (Wt. %) |
| 1 | Talc and Triethoxycaprylylsilane | 23.4 |
| 2 | Mica and Triethoxycaprylylsilane | 35.1 |
| 3 | Titanium Dioxide, Alumina, and Methicone | 12.2 |
| 4 | Zinc Oxide and Triethoxycaprylylsilane | 3.2 |
| 5 | Boron Nitride | 4.3 |
| 6 | CI 77492 (Yellow Iron Oxide) and Isopropyl | 2.3 |
| | Titanium Triisostearate | |
| 7 | CI 77491 (Red Iron Oxide) and Isopropyl | 0.6 |
| | Titanium Triisostearate | |
| 8 | CI 77499 (Black Iron Oxide) and Isopropyl | 0.4 |
| | Titanium Triisostearate | |
| 9 | Dimethicone and Trimethylsiloxysilicate | 2.1 |
| 10 | Caprylic/Capric Triglyceride | 2.8 |
| 11 | Sorbitan Sesquiisostearate | 0.5 |
| 12 | Diisostearyl Malate | 0.5 |
| 13 | Phenoxyethanol | 0.3 |
| 14 | Ethylhexyl Methoxycinnamate | 2.1 |
| 15 | Spherical PHBV particles from Example 4 | 10.0 |

### Formulation 3: Emulsified Foundation

Using the above spherical PHB particles from Example 1, an emulsified foundation with the ingredients in TABLE 6 was prepared. The preparation method was as follows: first, ingredients (5) to (8) were mixed with a IKA mixer, then ingredients (1) and (3) to (4) were added thereto, and the mixture was thoroughly mixed, and then pulverized and classified; further, ingredients (2) and (9) to (18) were added, treated with a homomixer, deaerated, and filled in a container.

TABLE 6
Emulsified Foundation Formulation

| Ingredient | | Amount |
|---|---|---|
| No. | Ingredient | (Wt. %) |
| 1 | Spherical PHB particles from Example 1 | 10.0 |
| 2 | Dimethyl silicone (viscosity 10 mPa · s) | 7.0 |
| 3 | Titanium oxide | 5.0 |
| 4 | Anhydrous silicic acid | 3.0 |
| 5 | Talc | 8.0 |
| 6 | Bengala | 1.0 |
| 7 | Black iron oxide | 0.5 |
| 8 | Yellow iron oxide | 1.0 |
| 9 | Octamethylcyclotetrasiloxane | 10.0 |
| 10 | Rosin pentaerislit ester | 2.0 |
| 11 | Neopentyl glycol diisooctanoate | 4.0 |
| 12 | Squalene | 2.5 |
| 13 | Glycerin triisooctanoate | 2.0 |
| 14 | Purified water | 35.0 |
| 15 | 1,3-butylene glycol | 4.0 |
| 16 | Ethanol | 8.0 |
| 17 | Preservatives | 0.1 |
| 18 | Fragrance | Balance |

### Formulation 4: Powder Foundation

Using the above spherical PHBV particles from Example 4, a dual-purpose powder foundation with the ingredients in TABLE 7 was prepared. The preparation method was as follows: first, ingredients (1) and (3) to (10) were mixed, pulverized, and transferred to an IKA mixer; and then ingredients (2) and (11) to (16) were added and mixed uniformly. The resulting mixture was compression molded into a metal plate.

TABLE 7
Powder Foundation Formulation

| Ingredient | | Amount |
|---|---|---|
| No. | Ingredient | (Wt. %) |
| 1 | Spherical PHBV particles from Example 4 | 15.0 |
| 2 | Dimethyl silicone (viscosity 10 mPa · s) | 5.0 |
| 3 | Mica | 5.0 |
| 4 | Talc | 5.0 |
| 5 | Titanium oxide | 15.0 |
| 6 | Mica Titanium | 3.5 |
| 7 | Iron oxides (red, yellow, and black) | 7.0 |
| 8 | Zinc oxide | 4.0 |
| 9 | Aluminum oxide | 10.0 |
| 10 | Barium sulfate | 5.0 |
| 11 | Lanolin | 5.0 |
| 12 | Vaseline | 1.5 |
| 13 | Liquid paraffin | 1.0 |
| 14 | Isopropyl millistate | 1.5 |
| 15 | Preservatives | Balance |
| 16 | Fragrance | Balance |

### Formulation 5: Powder Eyeshadow

Using the above spherical PHB particles from Example 1, a powder eyeshadow with the ingredients in TABLE 8 was prepared. The preparation method was as follows: first, ingredients (1) and (3) to (10) were mixed, crushed, and transferred to a Henschel mixer; then ingredients (2) and (11) were added and mixed uniformly. The mixture was compression molded into a metal plate.

TABLE 8
Powder Eyeshadow Formulation

| Ingredient | | Amount |
|---|---|---|
| No. | Ingredient | (Wt. %) |
| 1 | Spherical PHB particles from Example 1 | 20.0 |
| 2 | Dimethyl silicone (viscosity 10 mPa · s) | 5.0 |
| 3 | Mica | 1.0 |
| 4 | Talc | 15.0 |
| 5 | Mica Titanium | 8.0 |
| 6 | Zinc stearate | 4.0 |
| 7 | Zinc laurate | 4.0 |
| 8 | Yellow iron oxide | 0.7 |
| 9 | Black iron oxide | 0.7 |
| 10 | Red iron oxide | 0.7 |
| 11 | Liquid paraffin | 8.0 |
| 12 | Preservatives and fragrances | Balance |

### Formulation 6: Two-Layer Separation Type Sunscreen

Using the above spherical PHIB particles from Example 3, a two-layer separation type sunscreen emulsion with the ingredients in TABLE 9 was prepared. In the preparation method, ingredients (1) to (7) were first dispersed and mixed with a homogenizer. Then the aqueous phase ingredients (8) to (11) were added and stirred to emulsify.

TABLE 9
Two-Layer Sunscreen Formulation

| Ingredient | | Amount |
|---|---|---|
| No. | Ingredient | (Wt. %) |
| 1 | Spherical PHB particles from Example 3 | 10.0 |
| 2 | Dimethyl silicone (viscosity 10 mPa · s) | 5.0 |
| 3 | Decamethylcyclopentasiloxane | 20.0 |
| 4 | Polyether-modified silicone | 1.0 |
| 5 | Squalene | 8.0 |
| 6 | Hydrophobicized titanium oxide | 5.0 |
| 7 | Octyl methoxycinnamic acid | 7.0 |
| 8 | Glycerin | 2.0 |
| 9 | Sodium Chloride | 0.40 |
| 10 | Polysorbate 20 | 0.90 |
| 11 | Ethanol | 5.0 |
| 12 | Fragrance | 0.1 |
| 13 | Purified water | Balance |

### Formulation 7: Sunscreen Cream

Using the above spherical PHBV particles from Example 4, a sunscreen cream with the ingredients in TABLE 10 was prepared. In the preparation method, ingredients (1) to (8) were first dispersed and mixed with a homogenizer. Then the aqueous phases ingredients (8) to (10) were added and stirred to emulsify.

TABLE 10
Sunscreen Cream Formulation

| Ingredient | | Amount |
|---|---|---|
| No. | Ingredient | (Wt. %) |
| 1 | Spherical PHBV particles from Example 4 | 10.0 |
| 2 | Dimethyl silicone (viscosity 10 mPa · s) | 7.0 |
| 3 | Hydrophobic titanium dioxide | 10.0 |
| 4 | Hydrophobic zinc oxide | 10.0 |
| 5 | Squalene | 15.0 |
| 6 | Glycerin diisostearate | 3.0 |
| 7 | Preservatives | 0.1 |
| 8 | Fragrance | 0.1 |
| 9 | Purified water | Balance |
| 10 | 1,3-butylene glycol | 5.0 |

### Formulation 8: Solid White Powder

Using the above spherical PHB particles from Example 2, a solid white powder with the ingredients in TABLE 11 was prepared. Preparation method is as below: first, the ingredients (1) and (3) to (6) were mixed, crushed, and transferred to a Henschel mixer; then ingredients (2) and (7) to (10) were added, mixed uniformly, and compression-molded into a metal plate.

TABLE 11
Solid White Powder Formulation

| Ingredient | | Amount |
|---|---|---|
| No. | Ingredient | (Wt. %) |
| 1 | Spherical PHB particles from Example 2 | 20.0 |
| 2 | Dimethyl silicone (viscosity 10 mPa · s) | 7.0 |
| 3 | Mica Balance | 1.0 |
| 4 | Talc | 15.0 |
| 5 | Titanium oxide | 1.0 |
| 6 | Yellow iron oxide | 1.0 |
| 7 | Liquid paraffin | 10.0 |
| 8 | Beeswax | 3.0 |
| 9 | Preservatives | 0.1 |
| 10 | Fragrance | Balance |

### Formulation 9: Blusher

Using the above spherical PHB particles from Example 1, a blusher with the ingredients in TABLE 12 was prepared. The preparation method was as follows: first, the ingredients (1) to (6) were mixed, crushed, and transferred to a IKA mixer; then the ingredients (7) to (10) were added, mixed uniformly, and compression molded into a metal plate.

TABLE 12
Blusher Formulation

| Ingredient | | Amount |
|---|---|---|
| No. | Ingredient | (Wt. %) |
| 1 | Spherical PHB particles from Example 1 | 5.0 |
| 2 | Mica | 10.0 |
| 3 | Titanium oxide | 10.0 |
| 4 | Red iron oxide | 1.5 |
| 5 | Black iron oxide | 1.5 |
| 6 | Yellow iron oxide | 1.5 |
| 7 | Squalene | 7.0 |
| 8 | Dimethyl silicone (viscosity 5 mPa · s) | 7.0 |
| 9 | Preservatives | 0.1 |
| 10 | Fragrance | Balance |

### Formulation 10: Lipstick

Using the above spherical PHB particles from Example 3, a lipstick with the ingredients in TABLE 13 was prepared. The preparation method was as follows: first, ingredients (1)-(11) were heated and melted; then ingredients (12) and (13) were added and mixed. The resulting mixture was degassed and poured into a container, rapidly cooled, and hardened.

TABLE 13
Lipstick Formulation

| Ingredient | | Amount |
|---|---|---|
| No. | Ingredient | (Wt. %) |
| 1 | Spherical PHB particles from Example 3 | 6.0 |
| 2 | Dimethyl silicone (viscosity 10 mPa · s) | 10.0 |
| 3 | Paraffin wax | 11.0 |
| 4 | Lanolin wax | 12.0 |
| 5 | Candelilla wax | 5.0 |
| 6 | Beeswax | 5.0 |
| 7 | Castor oil | 2.0 |
| 8 | Glycerin trioctanoate | 2.0 |
| 9 | Titanium oxide | 1.0 |
| 10 | Red No. 201 | 3.0 |
| 11 | Blue No. 1 aluminum lake | 0.5 |
| 12 | Preservatives | 0.1 |
| 13 | Fragrance | Balance |

### Formulation 11: BB Cream

Using the above spherical PHBV particles from Example 4, a BB cream with the ingredients in TABLE 14 was prepared. The preparation method was as follows: ingredients (1) to (9) were mixed until uniform; ingredients (10) to (15) were separately mixed well, then added to ingredients (1) to (9) the combined ingredients were well mixed; then the aqueous phases ingredients (16) to (21) were added and mixed well; finally ingredient (22) was added and the resulting mixture was homogenized to uniform.

TABLE 14
BB Cream Formulation

| Ingredient | | Amount |
|---|---|---|
| No. | Ingredients | (Wt. %) |
| 1 | Caprylyl Methicone and C30-45 Alkyl | 4 |
| | Cetearyl Dimethicone Crosspolymer | |
| 2 | Dimethicone | 15 |
| 3 | Spherical PHBV particles from Example 4 | 5 |
| 4 | Trifluoropropyldimethylsiloxy/ | 2 |
| | Trimethylsiloxy Silsesquioxane and | |
| | Dimethicone | |
| 5 | (Caprylic/Capric) Triglyceride and | 7 |
| | PEG/PPG-20/15 Dimethicone | |
| 6 | Bisphenylpropyl Dimethicone | 2 |
| 7 | Boron Nitride | 3 |
| 8 | Phenoxyethanol (and) Ethylhexylglycerin | 1 |
| 9 | Fragrance | 0.1 |
| 10 | Caprylyl Methicone | 5 |
| 11 | Titanium Dioxide and Triethoxycaprylylsilane | 2 |
| 12 | Iron Oxide and Triethoxycaprylylsilane | 0.5 |
| 13 | Iron Oxide and Triethoxycaprylylsilane | 0.2 |
| 14 | Iron Oxide and Triethoxycaprylylsilane | 0.05 |
| 15 | Titanium Dioxide (and) Alumina and Methicone | 7 |
| 16 | Water | Balance |
| 17 | Trisodium EDTA | 0.2 |
| 18 | Butylene Glycol | 3 |
| 19 | Glycerin | 5 |
| 20 | Sodium Chloride | 1 |
| 21 | Hydroxyphenyl Propamidobenzoic Acid | 1 |
| 22 | Disteardimonium Hectorite | 0.7 |

### Formulation 12: CC Cream

Using the above spherical PHB particles from Example 3, a CC cream with the ingredients in TABLE 15 was prepared. The preparation method was as follows: ingredients (5) to (14) were mixed until uniform; ingredients (15) to (17) were separately mixed well, then added to ingredients (5) to (14). The combined ingredients were well mixed. Then the aqueous phase ingredients (1) to (4) were added and mixed well. Finally ingredients (18) to (20) were added and the resulting mixture was mixed to uniform.

TABLE 15
CC Cream Formulation

| Ingredient | | Amount |
|---|---|---|
| No. | Ingredients | (Wt. %) |
| 1 | Water | Balance |
| 2 | Glycerin | 5 |
| 3 | Sodium Chloride | 1 |
| 4 | Propylene Glycol | 8 |
| 5 | PEG-9 Dimethicone | 1 |
| 6 | Caprylic/Capric Triglyceride and | 3 |
| | PEG/PPG-20/15 Dimethicone | |
| 7 | Caprylyl Methicone | 12 |
| 8 | Diphenyl Dimethicone | 5 |
| 9 | Boron Nitride | 1 |
| 10 | Spherical PHB particles from Example 3 | 5 |
| 11 | Polymethylsilsesquioxane | 1 |
| 12 | Octyl MethoxyCinnamate | 4 |
| 13 | Titanium Dioxide and | 5 |
| | Methylhydrogenpolysiloxane | |
| 14 | Disteardimonium Hectorite | 0.5 |
| 15 | Yellow Iron Oxide and | 0.25 |
| | Methylhydrogenpolysiloxane | |
| 16 | Red Iron Oxide and | 0.15 |
| | Methylhydrogenpolysiloxane | |
| 17 | Black Iron Oxide and | 0.08 |
| | Methylhydrogenpolysiloxane | |
| 18 | Dimethicone/Vinyl Dimethicone | 5 |
| | Crosspolymer and | |
| | Dimethicone and Isohexadecane and | |
| | Cetearyl Methicone | |
| 19 | Fragrance | 0.1 |
| 20 | Phenoxyethanol and Chlorphenesin and | Balance |
| | Glycerin | |

### Formulation 13: Mascara

Using the above spherical PHB particles from Example 3, a Mascara with the ingredients in TABLE 16 was prepared. The preparation method was as follows: ingredients (3) and (4) were mixed in water then heated to uniform; then ingredients (2) and (5) to (9) were added and mixed well. Ingredients (10) to (15) were heated separately, added to the mixture, mixed well, and cooled to 45° C. Ingredients (16) and (17) were added individually, and the resulting mixtures was mixed to uniformity.

TABLE 16
Mascara Formulation

| Ingredient | | Amount |
|---|---|---|
| No. | Ingredients | (Wt. %) |
| 1 | Water | Balance |
| 2 | Spherical PHB particles from Example 3 | 5 |
| 3 | Polyvinylpyrrolidone | 2 |
| 4 | Hydroxyethylcellulose | 1 |
| 5 | Triethanolamine | 1 |
| 6 | Methylparaben | 0.3 |
| 7 | Disodium EDTA | 0.1 |
| 8 | Black Iron Oxide | 10 |
| 9 | Dimethicone PEG-8 Polyacrylate | 7.2 |
| 10 | Stearic Acid | 4.5 |
| 11 | Glyceryl Monostearate | 2 |
| 12 | White Bleached Beeswax | 7 |
| 13 | Carnauba Wax | 4.5 |
| 14 | Hydroxylated Lanolin | 1 |
| 15 | Propylparaben | Balance |
| 16 | Acrylates Copolymer | 20 |
| 17 | DMDM Hydantoin | Balance |

### Formulation 14: Concealer

Using the above spherical PHBV particles from Example 4, a concealer with the ingredients in TABLE 17 was prepared. The preparation method was as follows: ingredients (1) and (7) were mixed until uniform and heated to 90° C.; then ingredients (8) to (10) were added and mixed until uniform. The resulting mixture was poured into a suitable container.

TABLE 17
Concealer Formulation

| Ingredient | | Amount |
|---|---|---|
| No. | Ingredients | (Wt. %) |
| 1 | Boron Nitride | 10 |
| 2 | Spherical PHBV particles from Example 4 | 5 |
| 3 | Dimethicone | Balance |
| 4 | Titanium Dioxide andTriethoxycaprylylsilane | 4 |
| 5 | Iron Oxide and Triethoxycaprylylsilane | 0.9 |
| 6 | Iron Oxide and Triethoxycaprylylsilane | 0.3 |
| 7 | Iron Oxide and Triethoxycaprylylsilane | 0.1 |
| 8 | Ozokerite Wax | 5 |
| 9 | Polyethylene | 5 |
| 10 | Synthetic Wax and Microcrystalline Wax | 2 |

### Formulation 15: O/W Cream

Using the above spherical PHB particles from Example 1, an O/W Cream with the ingredients in TABLE 18 was prepared. The preparation method was as follows: ingredients (1) to (3) were mixed and heated to 80° C.; ingredients (4) to (6) were mixed separately and added to ingredients (1) to (3), mixed until uniform, and cooled to room temperature; then ingredients (7) and (8) were added, and the resulting mixture was mixed until uniform.

TABLE 18
O/W Cream Formulation

| Ingredient | | Amount |
|---|---|---|
| No. | Ingredients | (Wt. %) |
| 1 | Water | Balance |
| 2 | Spherical PHB particles from Example 1 | 9.5 |
| 3 | Titanium Dioxide, Silica, Aluminum Hydroxide | 0.5 |
| 4 | Polysilicone-34 and Isononyl Isononanoate and | 3 |
| | Water | |
| 5 | Dimethicone | 8 |
| 6 | Butyrospermum Parkii (Shea) Butter | 2 |
| 7 | Phenoxyethanol | Balance |
| 8 | Sodium Hyaluronate | 0.1 |

### Formulation 16: W/O Cream

Using the above spherical PHB particles from Example 3, a W/O cream with the ingredients in TABLE 19 was prepared. The preparation method was as follows: ingredients (1) to (6) were mixed and heated to 70° C.; ingredients (7) to (9) were mixed separately and added to ingredients (1) to (6). The resulting mixture was mixed to emulsify, and cooled to room temperature.

TABLE 19
W/O Cream Formulation

| Ingredient | | Amount |
|---|---|---|
| No. | Ingredients | (Wt. %) |
| 1 | Dimethicone | 1 |
| 2 | Capylyl Methicone | 10 |
| 3 | Spherical PHB particles from Example 3 | 9 |
| 4 | Hydrogen Dimethicone and Titanium | 1 |
| | Dioxide and Aluminum Hydroxide | |
| 5 | (Caprylyl/Capryl) Triglyceride and | 3 |
| | PEG/PPG-20/15 Dimethicone | |
| 6 | Phenoxy ethanol | Balance |
| 7 | Water | Balance |
| 8 | Glycerin | 8 |
| 9 | Sodium Chloride | 1 |

### Formulation 17: Eye Cream

Using the above spherical PHB particles from Example 3, an eye cream with the ingredients in TABLE 20 was prepared. The preparation method was as follows: ingredients (1) to (4) were mixed and heated to 70° C.; ingredients (5) to (12) were mixed separately and heated to 70° C.; then added to ingredients (1) to (4). The resulting mixture was mixed to emulsify, and cooled to 50° C. Then ingredient (13) was added and mixed until uniform.

TABLE 20
Eye Cream Formulation

| Ingredient | | Amount |
|---|---|---|
| No. | Ingredients | (Wt. %) |
| 1 | Water | Balance |
| 2 | Spherical PHB particles from Example 3 | 5 |
| 3 | Glycerin | 3 |
| 4 | Disodium EDTA | 0.05 |
| 5 | Glyceryl stearate citrate and Polyglyceryl- | 4 |
| | 3 stearate and Hydrogenated lecithin | |
| 6 | Sodium acrylates copolymer and Lecithin | 1 |
| 7 | Cetyl Alcohol | 1.5 |
| 8 | Butyrospermum Parkii (Shea Butter) | 4 |
| 9 | Decyl Isostearate and Isostearyl Isostearate | 3 |
| 10 | Argania Spinosa Kernel Oil | 2 |
| 11 | Dimethicone | 3 |
| 12 | Caprylyl Methicone | 3 |
| 13 | Dimethicone/Vinyl Dimethicone Crosspolymer | 3 |
| | and Dimethicone and Isohexadecane and | |
| | Cetearyl Methicone | |

### Formulation 18: Skin Serum

Using the above spherical PHBV particles from Example 4, a skin serum with the ingredients in TABLE 21 was prepared. The preparation method was as follows: ingredients (1) to (4) were mixed until uniform; ingredients (5) to (7) were mixed separately, added to ingredients (1) to (4), and mixed until uniform; then ingredients from (8) to (11) were added one by one, and mixed until uniform.

TABLE 21
Skin Serum Formulation

| Ingredient | | Amount |
|---|---|---|
| No. | Ingredients | (Wt. %) |
| 1 | Water | Balance |
| 2 | Spherical PHBV particles from Example 4 | 1 |
| 3 | Alpha-Glucan Oligosaccharide | 0.5 |
| 4 | Hydrolyzed Sodium Hyaluronate | 0.01 |
| 5 | BG | 1 |
| 6 | Pentylene Glycol | 3 |
| 7 | Dipropylene Glycol and Polysilicone-29 | 1 |
| 8 | Malus Domestica Fruit Cell Culture Extract | 0.1 |
| 9 | Ascorbyl Tetraisopalmitate | 0.1 |
| 10 | Phenoxyethanol | Balance |
| 11 | Fragrance | Balance |

### Formulation 19: Shampoo

Using the above spherical PHB particles from Example 2, a shampoo with the ingredients in TABLE 22 was prepared. The preparation method was as follows: ingredients (3) was mixed with water until uniform; then the mixture was heated to 80° C. After the mixture was clear, ingredients (2) and (5) to (10) were added to the mixture one by one. After the mixture became clear again, the mixture was cooled to room temperature. Then ingredients (11) to (15) were added one by one, and mixed until uniform.

TABLE 22
Shampoo Formulation

| Ingredient | | Amount |
|---|---|---|
| No. | Ingredients | (Wt. %) |
| 1 | Water | Balance |
| 2 | Dipropylene Glycol | 3 |
| 3 | Polyquaternium 10 | 0.4 |
| 5 | Spherical PHB particles from Example 2 | 5 |
| 6 | Disodium EDTA | 0.05 |
| 7 | Sodium Benzoate | 0.5 |
| 8 | Sodium Laureth Sulfate | 17 |
| 9 | Cocamidopropyl Betaine | 10 |
| 10 | Cocamide MEA | 2 |
| 11 | Phenoxyethanol | Balance |
| 12 | Water, Glycol Distearate, Glycerin, | 5 |
| | Laureth-4, Cocamidopropyl Betain | |
| 13 | Citric acid | 0.1 |
| 14 | Dimethiconol, Water, Sodium Lauryl | 2 |
| | Sulfate, Sodium Laureth Sulfate | |
| 15 | Fragrance | Balance |

### Formulation 20: Skin Primer

Using the above spherical PHB particles from Example 3, a skin primer with the ingredients in TABLE 23 was prepared. The preparation method was as follows: ingredients (1) to (7) were heated and mixed until uniform; ingredients (8) to (13) were separately mixed until uniform, then added to ingredients (1) to (7). The resulting mixture was well mixed. Finally the aqueous phase ingredients (14) to (18) were added and stirred to emulsify.

TABLE 23
Skin Primer Formulation

| Ingredient | | Amount |
|---|---|---|
| No. | Ingredients | (Wt. %) |
| 1 | Dimethicone | 10.5 |
| 2 | Ethylhexyl Methoxycinnamate | 5 |
| 3 | Spherical PHB particles from Example 3 | 5 |
| 4 | Cylcopentasiloxane and | 4 |
| | Polymethylsilsesquioxane | |
| 5 | Dimethicone, Cetearyl Dimethicone | 4 |
| | Crosspolymer | |
| 6 | Sorbitan Sesquiisostearate | 0.5 |
| 7 | (Caprylic/Capric) Triglyceride and | 4 |
| | PEG/PPG-20/15 Dimethicone | |
| 8 | Dimethicone | 3 |
| 9 | Hydrogen Dimethicone and Zinc Oxide | 1.5 |
| 10 | Hydrogen Dimethicone and Titanium | 1.5 |
| | Dioxide (and) Aluminum Hydroxide | |
| 11 | Methicone and Yellow Iron Oxide (CI 77492) | 0.05 |
| 12 | Methicone and Red Iron Oxide (CI 77491) | 0.05 |
| 13 | Methicone and Black Iron Oxide (CI 77499) | 0.015 |
| 14 | water | Balance |
| 15 | Glycerin | 5 |
| 16 | Polysorbate 20 | 0.5 |
| 17 | Sodium Chloride | 1 |
| 18 | Phenoxy ethanol | Balance |

### Other Aspects

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

1. A composition comprising polyhydroxyalkanoate (PHA) particles,
wherein the PHA particles comprise at least one PHA polymer and wherein the
PHA particles have a sphericity of at least 0.95, a solidity of at least 90%, and
a Dv50 particle size from about 1 μm to about 50 μm.

2. The composition of claim 1, wherein the PHA polymer is a compound of Formula (I): 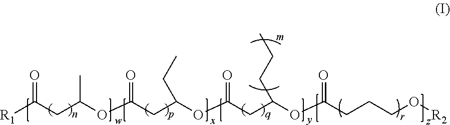
wherein:
w, x, y, and z are each independently an integer from 0 to 5000, wherein at least one of w, x, y, and z is ≥1;
m, n, p, q, and r are each independently an integer from 1 to 10; and
R₁ and R₂ are each independently H or CH₃.

3. The composition of claim 1, wherein the at least one PHA polymer has a Mw from about 500 g/mol to about 5,000,000 g/mol.

4. The composition of claim 1, wherein the at least one PHA polymer is selected from the group consisting of poly-3-hydroxybutyrate (PHB), poly-3-hydroxyvalerate (PHV), poly-3-hydroxyhexanoate (PHH), poly-3-hydroxyheptanoate, poly-3-hydroxyoctanoate, poly-3-hydroxynonanoate, poly-4-hydroxybutyrate (P4HB), poly(3-hydroxybutyrate-co-3-hydroxyvalerate) (PHBV), poly(3-hydroxybutyrate-co-3-hydroxyhexanoate) (PHBH), poly(3-hydroxybutyrate-co-4-hydroxybutyrate), poly(3-hydroxybuytrate-co-3-hydroxyvalerate-co-3-hydroxyhexanoate), poly-3-hydroxybutyrate, and poly(3-hydroxybutyrate-co-3-hydroxyvalerate).

5. The composition of claim 1, wherein the at least one PHA polymer is poly-3-hydroxybutyrate or poly(3-hydroxybutyrate-co-3-hydroxyvalerate).

6. The composition of claim 1, wherein the PHA particles have a Dv50 particle size from about 1 μm to about 20 μm.

7. The composition of claim 1, wherein the PHA particles have a solidity of at least 95%.

8. The composition of claim 1, wherein the PHA particles have a sphericity of at least 0.99.

9. The composition of claim 1, wherein the PHA particles have a particle size coefficient of variance greater than 40.5%.

10. A personal care composition comprising the composition of PHA particles of claim 1.

11. The personal care composition of claim 10, further comprising at least one additional component selected from the group consisting of pigments, particulates, dyes, and combinations thereof.

12. The personal care composition of claim 11, wherein the additional component is selected from the group consisting of iron oxides, zinc oxides, titanium dioxides, mica, silica, tapioca starch, rice starch, cellulose, cellulose acetate, polymethylsilsesquioxane, mica, and combinations thereof.

13. A personal care formulation comprising the composition of PHA particles of claim 1, wherein the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

14. Use of the personal care composition of claim 10 in the manufacture of a personal care formulation.
